SEQUENCE LISTING (55 sequences)

SEQ ID NO: 1 - protein, 8 aa, synthetic construct
  REGION 1..8: note = Chi99H4D5/Hu99H4D5-V6 HCDR1 (IMGT)
GFTFSYYA

SEQ ID NO: 2 - protein, 7 aa, synthetic construct
  REGION 1..7: note = Chi99H4D5 HCDR2 (IMGT)
ISGNGDT

SEQ ID NO: 3 - protein, 7 aa, synthetic construct
  REGION 1..7: note = Chi99H4D5 HCDR2-V1 (IMGT); Chi70D3A6 HCDR2 (IMGT)
ISGGGDT

SEQ ID NO: 4 - protein, 7 aa, synthetic construct
  REGION 1..7: note = Chi99H4D5 HCDR2-V2 (IMGT)
ISGNGDV

SEQ ID NO: 5 - protein, 7 aa, synthetic construct
  REGION 1..7: note = Chi99H4D5 HCDR2-V3 (IMGT); Chi70D3A6 HCDR2-V1 (IMGT);Hu70D3A6-V12 HCDR2 (IMGT) Hu99H4D5-V6 HCDR2 (IMGT)
ISGGGDV

SEQ ID NO: 6 - protein, 11 aa, synthetic construct
  REGION 1..11: note = Chi99H4D5/ Hu70D3A6-V12/Hu99H4D5-V6 HCDR3 (IMGT)
ARGVGGNYFDY

SEQ ID NO: 7 - protein, 117 aa, synthetic construct
  REGION 1..117: note = Chi99H4D5 VH
EVKLVESGGGLVKPGGSLKLSCAASGFTFSYYAISWLRQTPEKRLEWVASISGNGDTYYLDSVKGRFTISRDNARNILYLQMSSLRSEDTAMYYCARGVGGNYFDYWGQGTTLTVSS

SEQ ID NO: 8 - protein, 117 aa, synthetic construct
  REGION 1..117: note = Chi99H4D5 VH-V1
EVKLVESGGGLVKPGGSLKLSCAASGFTFSYYAISWLRQTPEKRLEWVASISGGGDTYYLDSVKGRFTISRDNARNILYLQMSSLRSEDTAMYYCARGVGGNYFDYWGQGTTLTVSS

SEQ ID NO: 9 - protein, 117 aa, synthetic construct
  REGION 1..117: note = Chi99H4D5 VH-V2
EVKLVESGGGLVKPGGSLKLSCAASGFTFSYYAISWLRQTPEKRLEWVASISGNGDVYYLDSVKGRFTISRDNARNILYLQMSSLRSEDTAMYYCARGVGGNYFDYWGQGTTLTVSS

SEQ ID NO: 10 - protein, 117 aa, synthetic construct
  REGION 1..117: note = Chi99H4D5 VH-V3
EVKLVESGGGLVKPGGSLKLSCAASGFTFSYYAISWLRQTPEKRLEWVASISGGGDVYYLDSVKGRFTISRDNARNILYLQMSSLRSEDTAMYYCARGVGGNYFDYWGQGTTLTVSS

SEQ ID NO: 11 - protein, 5 aa, synthetic construct
  REGION 1..5: note = Chi99H4D5 LCDR1 (IMGT); Chi70D3A6 LCDR1 (IMGT); Hu70D3A6-V12 LCDR1 (IMGT); Hu99H4D5-V6 LCDR1 (IMGT)
SSVSY

SEQ ID NO: 12 - skipped

SEQ ID NO: 13 - protein, 10 aa, synthetic construct
  REGION 1..10: note = Chi99H4D5/Hu99H4D5-V6 LCDR3 (IMGT); Chi99H4D5/Hu99H4D5-V6 LCDR3 (AbM);Chi99H4D5/Hu99H4D5-V6 LCDR3 (Kabat);
QQYHSYTPIT

SEQ ID NO: 14 - protein, 107 aa, synthetic construct
  REGION 1..107: note = Chi99H4D5 VL
DIVMTQSPAIMSASPGEKVTISCSASSSVSYMYWYQQKPGSSPKPLIYRTSNLASGVPARFSGSGSGTSYSLTISTMEAEDAATYYCQQYHSYTPITFGAGTKLDLK

SEQ ID NO: 15 - protein, 8 aa, synthetic construct
  REGION 1..8: note = Chi70D3A6/ Hu70D3A6-V12 HCDR1 (IMGT)
GFTFTYYA

SEQ ID NO: 16 - protein, 11 aa, synthetic construct
  REGION 1..11: note = Chi70D3A6 HCDR3 (IMGT)
ARGVGGNFFDY

SEQ ID NO: 17 - protein, 117 aa, synthetic construct
  REGION 1..117: note = Chi70D3A6 VH
EVKLVESGGDLVKPGGSLKLSCTASGFTFTYYAMSWVRQTPEKRLEWVATISGGGDTYYLDSVKGRFTISRDNAGNILYLQMSSLRSEDTAMYYCARGVGGNFFDYWGQGSTLTVSS

SEQ ID NO: 18 - protein, 117 aa, synthetic construct
  REGION 1..117: note = Chi70D3A6 VH-V1
EVKLVESGGDLVKPGGSLKLSCTASGFTFTYYAMSWVRQTPEKRLEWVATISGGGDVYYLDSVKGRFTISRDNAGNILYLQMSSLRSEDTAMYYCARGVGGNFFDYWGQGSTLTVSS

SEQ ID NO: 19 - protein, 10 aa, synthetic construct
  REGION 1..10: note = Chi70D3A6 LCDR3 (IMGT);Chi70D3A6 LCDR3 (AbM); Chi70D3A6 LCDR3 (Kabat);Hu70D3A6-V12 LCDR3 (AbM);Hu70D3A6-V12 LCDR3 (Kabat);Hu70D3A6-V12 LCDR3 (IMGT); Hu70D3A6-V12LCDR3 (IMGT)
QLYHSYTPIT

SEQ ID NO: 20 - protein, 107 aa, synthetic construct
  REGION 1..107: note = Chi70D3A6 VL
QIVLTQSPAIMSASPGEKVTISCSASSSVSYMYWYQQKPGSSPKPFIYRTSNLASGVPSRFSGSGSGTSYSLTVSTMEAEDAATYYCQLYHSYTPITFGAGTKLELK

SEQ ID NO: 21 - protein, 10 aa, synthetic construct
  REGION 1..10: note = Chi99H4D5/Hu99H4D5-V6 HCDR1 (AbM)
GFTFSYYAIS

SEQ ID NO: 22 - protein, 9 aa, synthetic construct
  REGION 1..9: note = Chi99H4D5 HCDR2 (AbM)
SISGNGDTY

SEQ ID NO: 23 - protein, 9 aa, synthetic construct
  REGION 1..9: note = Chi99H4D5 HCDR2-V1 (AbM)
SISGGGDTY

SEQ ID NO: 24 - protein, 9 aa, synthetic construct
  REGION 1..9: note = Chi99H4D5 HCDR2-V2 (AbM)
SISGNGDVY

SEQ ID NO: 25 - protein, 9 aa, synthetic construct
  REGION 1..9: note = Chi99H4D5-V3/Hu70D3A6-V12/Hu99H4D5-V6 HCDR2 (AbM)
SISGGGDVY

SEQ ID NO: 26 - protein, 9 aa, synthetic construct
  REGION 1..9: note = Chi99H4D5 HCDR3 (AbM); Chi99H4D5 HCDR3 (Kabat); Hu70D3A6-V12 HCDR3 (AbM); Hu70D3A6-V12 HCDR3 (Kabat); Hu99H4D5-V6 HCDR3 (AbM); Hu99H4D5-V6 HCDR3 (Kabat)
GVGGNYFDY

SEQ ID NO: 27 - protein, 10 aa, synthetic construct
  REGION 1..10: note = Chi99H4D5 LCDR1 (AbM); Chi70D3A6 LCDR1 (AbM); Chi99H4D5 LCDR1 (Kabat); Chi70D3A6 LCDR1 (Kabat);Hu70D3A6-V12 LCDR1 (AbM);Hu70D3A6-V12 LCDR1 (Kabat);Hu99H4D5-V6 LCDR1 (AbM);Hu99H4D5-V6 LCDR1 (Kabat)
SASSSVSYMY

SEQ ID NO: 28 - protein, 7 aa, synthetic construct
  REGION 1..7: note = Chi99H4D5 LCDR2 (AbM); Chi70D3A6 LCDR2(AbM); Chi99H4D5 LCDR2 (Kabat); Chi70D3A6 LCDR2(Kabat);Hu70D3A6-V12 LCDR2(AbM);Hu70D3A6-V12 LCDR2 (Kabat); Hu99H4D5-V6 LCDR2 (AbM);Hu99H4D5-V6 LCDR2 (Kabat)
RTSNLAS

SEQ ID NO: 29 - protein, 10 aa, synthetic construct
  REGION 1..10: note = Chi70D3A6 HCDR1/Hu70D3A6-V12 (AbM)
GFTFTYYAMS

SEQ ID NO: 30 - protein, 9 aa, synthetic construct
  REGION 1..9: note = Chi70D3A6 HCDR2 (AbM)
TISGGGDTY

SEQ ID NO: 31 - protein, 9 aa, synthetic construct
  REGION 1..9: note = Chi70D3A6 HCDR2-V1 (AbM)
TISGGGDVY

SEQ ID NO: 32 - protein, 9 aa, synthetic construct
  REGION 1..9: note = Chi70D3A6 HCDR3 (AbM); Chi70D3A6 HCDR3 (Kabat)
GVGGNFFDY

SEQ ID NO: 33 - protein, 5 aa, synthetic construct
  REGION 1..5: note = Chi99H4D5/Hu99H4D5-V6 HCDR1 (Kabat)
YYAIS

SEQ ID NO: 34 - protein, 16 aa, synthetic construct
  REGION 1..16: note = Chi99H4D5 HCDR2 (Kabat)
SISGNGDTYYLDSVKG

SEQ ID NO: 35 - protein, 16 aa, synthetic construct
  REGION 1..16: note = Chi99H4D5 HCDR2-V1 (Kabat)
SISGGGDTYYLDSVKG

SEQ ID NO: 36 - protein, 16 aa, synthetic construct
  REGION 1..16: note = Chi99H4D5 HCDR2-V2 (Kabat)
SISGNGDVYYLDSVKG

SEQ ID NO: 37 - protein, 16 aa, synthetic construct
  REGION 1..16: note = Chi99H4D5 HCDR2-V3 (Kabat)
SISGGGDVYYLDSVKG

SEQ ID NO: 38 - protein, 5 aa, synthetic construct
  REGION 1..5: note = Chi70D3A6/ Hu70D3A6-V12 HCDR1 (Kabat)
YYAMS

SEQ ID NO: 39 - protein, 16 aa, synthetic construct
  REGION 1..16: note = Chi70D3A6 HCDR2 (Kabat)
TISGGGDTYYLDSVKG

SEQ ID NO: 40 - protein, 16 aa, synthetic construct
  REGION 1..16: note = Chi70D3A6-V1 HCDR2 (Kabat)
TISGGGDVYYLDSVKG

SEQ ID NO: 41 - protein, 796 aa, Homo sapiens
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 42 - protein, 391 aa, Homo sapiens
AAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDETPFGVSVAVG

SEQ ID NO: 43 - protein, 8 aa, synthetic construct
  REGION 1..8: note = Chi99H4D5 HCDR1 (IMGT); Chi70D3A6 HCDR1 (IMGT)
  VARIANT 5: note = misc_feature - Xaa = Ser or Thr
GFTFXYYA

SEQ ID NO: 44 - protein, 7 aa, synthetic construct
  REGION 1..7: note = Chi99H4D5 HCDR2 (IMGT); Chi99H4D5 HCDR2-V1 (IMGT); Chi70D3A6 HCDR2 (IMGT); Chi99H4D5 HCDR2-V2 (IMGT); Chi99H4D5 HCDR2-V3 (IMGT); Chi70D3A6 HCDR2-V1 (IMGT)
  VARIANT 4: note = misc_feature - Xaa = Gly or Asn
  VARIANT 7: note = misc_feature - Xaa = Thr or Val
ISGXGDX

SEQ ID NO: 45 - protein, 11 aa, synthetic construct
  REGION 1..11: note = Chi99H4D5 HCDR3 (IMGT); Chi70D3A6 HCDR3 (IMGT)
  VARIANT 8: note = misc_feature - Xaa = Phe or Tyr
ARGVGGNXFDY

SEQ ID NO: 46 - protein, 117 aa, synthetic construct
  REGION 1..117: note = Chi99H4D5 VH; Chi99H4D5 VH-V1; Chi99H4D5 VH-V2; Chi99H4D5 VH-V3; Chi70D3A6 VH; Chi70D3A6 VH-V1
  VARIANT 10: note = misc_feature - Xaa = Asp or Gly
  VARIANT 23: note = misc_feature - Xaa = Ala or Thr
  VARIANT 30: note = misc_feature - Xaa = Ser or Thr
  VARIANT 34: note = misc_feature - Xaa = Ile or Met
  VARIANT 37: note = misc_feature - Xaa = Leu or Val
  VARIANT 50: note = misc_feature - Xaa = Ser or Thr
  VARIANT 54: note = misc_feature - Xaa = Gly or Asn
  VARIANT 57: note = misc_feature - Xaa = Thr or Val
  VARIANT 75: note = misc_feature - Xaa = Gly or Arg
  VARIANT 103: note = misc_feature - Xaa = Phe or Tyr
  VARIANT 111: note = misc_feature - Xaa = Ser or Thr
EVKLVESGGXLVKPGGSLKLSCXASGFTFXYYAXSWXRQTPEKRLEWVAXISGXGDXYYLDSVKGRFTISRDNAXNILYLQMSSLRSEDTAMYYCARGVGGNXFDYWGQGXTLTVSS

SEQ ID NO: 47 - protein, 10 aa, synthetic construct
  REGION 1..10: note = Chi99H4D5 LCDR3 (IMGT); Chi99H4D5 LCDR3 (AbM); Chi99H4D5 LCDR3 (Kabat); Chi70D3A6 LCDR3 (IMGT); Chi70D3A6 LCDR3 (AbM); Chi70D3A6 LCDR3 (Kabat)
  VARIANT 2: note = misc_feature - Xaa = Leu or Gln
QXYHSYTPIT

SEQ ID NO: 48 - protein, 107 aa, synthetic construct
  REGION 1..107: note = Chi99H4D5 VL; Chi70D3A6 VL
  VARIANT 1: note = misc_feature - Xaa = Asp or Gln
  VARIANT 4: note = misc_feature - Xaa = Leu or Met
  VARIANT 46: note = misc_feature - Xaa = Phe or Leu
  VARIANT 59: note = misc_feature - Xaa = Ala or Ser
  VARIANT 74: note = misc_feature - Xaa = Ile or Val
  VARIANT 89: note = misc_feature - Xaa = Leu or Gln
  VARIANT 105: note = misc_feature - Xaa = Asp or Glu
XIVXTQSPAIMSASPGEKVTISCSASSSVSYMYWYQQKPGSSPKPXIYRTSNLASGVPXRFSGSGSGTSYSLTXSTMEAEDAATYYCQXYHSYTPITFGAGTKLXLK

SEQ ID NO: 49 - protein, 16 aa, synthetic construct
  REGION 1..16: note = Hu70D3A6-V12 HCDR2 (Kabat)
SISGGGDVYYADSVKG

SEQ ID NO: 50 - protein, 117 aa, synthetic construct
  REGION 1..117: note = Hu70D3A6-V12 VH
EVQLVESGGGLVKPGGSLRLSCAASGFTFTYYAMSWVRQAPGKGLEWVSSISGGGDVYYADSVKGRFTISRDNAKNTLYLQMNSLRAEDTAVYYCARGVGGNYFDYWGQGTLVTVSS

SEQ ID NO: 51 - protein, 107 aa, synthetic construct
  REGION 1..107: note = Hu70D3A6-V12 VL
DIQMTQSPSSLSASVGDRVTITCSASSSVSYMYWYQQKPGKAPKPLIYRTSNLASGVPSRFSGSGSGTDFTLTISSLQPEDFATYYCQLYHSYTPITFGQGTKLEIK

SEQ ID NO: 52 - protein, 117 aa, synthetic construct
  REGION 1..117: note = Hu99H4D5-V6 VH
EVQLVESGGGLVKPGGSLRLSCAASGFTFSYYAISWVRQAPGKGLEWVSSISGGGDVYYADSVKGRFTISRDNAKNTLYLQMNSLRAEDTAVYYCARGVGGNYFDYWGQGTLVTVSS

SEQ ID NO: 53 - protein, 107 aa, synthetic construct
  REGION 1..107: note = Hu99H4D5-V6 VL
DIQMTQSPSSLSASVGDRVTITCSASSSVSYMYWYQQKPGKAPKPLIYRTSNLASGVPSRFSGSGSGTDFTLTISSLQPEDFATYYCQQYHSYTPITFGQGTKLEIK

SEQ ID NO: 54 - protein, 117 aa, synthetic construct
  REGION 1..117: note = Hu70D3A6-V12 VH; Hu99H4D5-V6 VH; Chi99H4D5 VH; Chi99H4D5 VH-V1; Chi99H4D5 VH-V2; Chi99H4D5 VH-V3; Chi70D3A6 VH; Chi70D3A6 VH-V1
  VARIANT 3: note = misc_feature - Xaa = Lys or Gln
  VARIANT 10: note = misc_feature - Xaa = Asp or Gly
  VARIANT 19: note = misc_feature - Xaa = Lys or Arg
  VARIANT 23: note = misc_feature - Xaa = Ala or Thr
  VARIANT 30: note = misc_feature - Xaa = Ser or Thr
  VARIANT 34: note = misc_feature - Xaa = Ile or Met
  VARIANT 37: note = misc_feature - Xaa = Leu or Val
  VARIANT 40: note = misc_feature - Xaa = Ala or Thr
  VARIANT 42: note = misc_feature - Xaa = Glu or Gly
  VARIANT 44: note = misc_feature - Xaa = Gly or Arg
  VARIANT 49: note = misc_feature - Xaa = Ala or Ser
  VARIANT 50: note = misc_feature - Xaa = Ser or Thr
  VARIANT 54: note = misc_feature - Xaa = Gly or Asn
  VARIANT 57: note = misc_feature - Xaa = Thr or Val
  VARIANT 60: note = misc_feature - Xaa = Ala or Leu
  VARIANT 75: note = misc_feature - Xaa = Gly, Lys or Arg
  VARIANT 77: note = misc_feature - Xaa = Ile or Thr
  VARIANT 83: note = misc_feature - Xaa = Asn or Ser
  VARIANT 87: note = misc_feature - Xaa = Ala or Ser
  VARIANT 92: note = misc_feature - Xaa = Met or Val
  VARIANT 103: note = misc_feature - Xaa = Phe or Tyr
  VARIANT 111: note = misc_feature - Xaa = Ser or Thr
  VARIANT 112: note = misc_feature - Xaa = Leu or Thr
  VARIANT 113: note = misc_feature - Xaa = Leu or Val
EVXLVESGGXLVKPGGSLXLSCXASGFTFXYYAXSWXRQXPXKXLEWVXXISGXGDXYYXDSVKGRFTISRDNAXNXLYLQMXSLRXEDTAXYYCARGVGGNXFDYWGQGXXXTVSS

SEQ ID NO: 55 - protein, 107 aa, synthetic construct
  REGION 1..107: note = Chi99H4D5 VL; Chi70D3A6 VL; Hu70D3A6-V12 VL; Hu99H4D5-V6 VL
  VARIANT 1: note = misc_feature - Xaa = Asp or Gln
  VARIANT 3: note = misc_feature - Xaa = Gln or Val
  VARIANT 4: note = misc_feature - Xaa = Leu or Met
  VARIANT 9: note = misc_feature - Xaa = Ala or Ser
  VARIANT 10: note = misc_feature - Xaa = Ile or Ser
  VARIANT 11: note = misc_feature - Xaa = Leu or Met
  VARIANT 15: note = misc_feature - Xaa = Pro or Val
  VARIANT 17: note = misc_feature - Xaa = Asp or Glu
  VARIANT 18: note = misc_feature - Xaa = Lys or Arg
  VARIANT 22: note = misc_feature - Xaa = Ser or Thr
  VARIANT 41: note = misc_feature - Xaa = Lys or Ser
  VARIANT 42: note = misc_feature - Xaa = Ala or Ser
  VARIANT 46: note = misc_feature - Xaa = Phe or Leu
  VARIANT 59: note = misc_feature - Xaa = Ala or Ser
  VARIANT 69: note = misc_feature - Xaa = Asp or Ser
  VARIANT 70: note = misc_feature - Xaa = Phe or Tyr
  VARIANT 71: note = misc_feature - Xaa = Ser or Thr
  VARIANT 74: note = misc_feature - Xaa = Ile or Val
  VARIANT 76: note = misc_feature - Xaa = Ser or Thr
  VARIANT 77: note = misc_feature - Xaa = Leu or Met
  VARIANT 78: note = misc_feature - Xaa = Glu or Gln
  VARIANT 79: note = misc_feature - Xaa = Ala or Pro
  VARIANT 82: note = misc_feature - Xaa = Ala or Phe
  VARIANT 89: note = misc_feature - Xaa = Leu or Gln
  VARIANT 100: note = misc_feature - Xaa = Ala or Gln
  VARIANT 105: note = misc_feature - Xaa = Asp or Glu
  VARIANT 106: note = misc_feature - Xaa = Ile or Leu
XIXXTQSPXXXSASXGXXVTIXCSASSSVSYMYWYQQKPGXXPKPXIYRTSNLASGVPXRFSGSGSGTXXXLTXSXXXXEDXATYYCQXYHSYTPITFGXGTKLXXK

(19) United States
(12) Patent Application Publication
(10) Pub. No.: US 2026/0365740 A1
(43) Pub. Date: Nov. 19, 2026

(54) TrkA ANTIBODY AND USE THEREOF

(71) Applicant: 4B Technologies (Shanghai) Co., Ltd, Shanghai (CN)

(72) Inventors: Gang LU, Suzhou, Jiangsu (CN); Jun ZHOU, Suzhou, Jiangsu (CN); Dong YANG, Suzhou, Jiangsu (CN); Jie XIE, Suzhou, Jiangsu (CN); Jing SU, Beijing (CN); Chen LV, Suzhou, Jiangsu (CN); Xiaoming GUAN, Suzhou, Jiangsu (CN)

(73) Assignee: 4B Technologies (Shanghai) Co., Ltd, Shanghai (CN)

(21) Appl. No.: 19/486,444

(22) PCT Filed: May 22, 2024
(86) PCT No.: PCT/CN2024/094591

§ 371 Date: Nov. 21, 2025

(30) Foreign Application Priority Data
May 23, 2023 (WO) PCT/CN2023/095660

(51) Int. Cl. C07K 16/28; A61P 25/28

(52) U.S. Cl. CPC C07K 16/2863; A61P 25/28

(57) ABSTRACT

The present disclosure provides TrkA agonistic antibodies, compositions comprising such antibodies, and methods of using such antibodies for the prevention and/or treatment of a disease or disorder.

### BACKGROUND OF THE INVENTION

Nerve growth factor (NGF) belongs to the neurotrophin family that regulates the cell proliferation, growth, survival and differentiation of neurons in both central and peripheral nervous systems in all vertebrate species.

NGF binds to its cognate tropomyosin-related kinase A (TrkA) receptor and the p75 pan-neurotrophin receptor (p75NTR). NGF binding to TrkA activates downstream survival pathways by activating Akt, and activates downstream differentiation pathways by activating ERK and PLCγ. p75NTR could be activated by pro-NGF or NGF to induce pain and cell death via apoptosis.

NGF/TrkA pathway plays a major role in the growth, maintenance, proliferation, and survival of sympathetic and small-diameter sensory neurons, thus accelerating the repair of nerve damage. A direct correlation was observed between the reduction of NGF levels in the skin and small fiber sensory neuropathy in diabetic patients. NGF has been extensively investigated for its potential to treat peripheral neuropathies such as chemotherapy-induced peripheral neuropathy (CIPN), and diabetic neuropathy in pre-clinical and in clinical settings. However, all clinical trials using NGF were discontinued mainly due to significant side effects, including injection site hyperalgesia, myalgia.

Nerve growth factor (NGF) and TrkA agonist have been demonstrated to stimulate corneal sensitivity, nerve regeneration and have been suggested as a potential treatment for ophthalmic diseases such as dry eye disease (DED). Activation of NGF-TrkA signaling stimulates conjunctival mucin secretion, thereby may stabilize the tear film. In addition, NGF improves corneal healing and may reduce ocular surface damage. Clinical studies confirmed the efficacy of rhNGF and TrkA agonists for dry eye disease. Nonetheless, eye pain/irritation is a major obstacle in clinical application of rhNGF for ophthalmic diseases, despite significant improvement on ocular damage.

Several studies have shown the correlation between NGF level and apoptosis of Langerhans islet cells. Decreased level of NGF induces apoptotic death in pancreatic β-cells by having an impact on metabolism. NGF might be a prospective therapeutic agent in diabetes and related complications.

In preclinical studies, NGF has been shown to improve cardiac function, decrease myocardial apoptosis and fibrosis, and protect sympathetic cardiac innervation against ischemia. Small molecular TrkA activator has also been shown to protect neuronal cell, as well as have therapeutic effect on heart disorders. However, short half-life time and injection site hyperalgesia have hampered the drug development.

Evidence from preclinical models of neuronal injury and aging support a pivotal role for NGF in neuroprotection, resilience, and cognitive function. TrkA agonist treatment effectively attenuated cholinergic degeneration and promoted functional recovery, resulted in reduction of brain amyloid pathology and dystrophic neurites surrounding amyloid plaques, and improved cognitive function in the model of Alzheimer's disease (AD). TrkA agonism is therefore a potential therapeutic target for AD.

TrkA agonistic antibody has been reported previously, which can mimick the neurotrophic function of NGF, but also share its side effects such as induction of hyperalgesia or pain. There is a significant unmet need to develop a NGF mimetics for the treatment of various diseases with reduced side effects such as pain.

### SUMMARY OF THE INVENTION

The present disclosure provides a TrkA antibody and use thereof. Particularly, the present application provides TrkA agonistic antibodies, compositions comprising such antibodies, and methods of using such antibodies for the treatment of disease and/or disorder, such as tumors, peripheral neuropathies, ophthalmic diseases, diabetes and related complications, cardiovascular disease and/or Alzheimer's disease.

In one aspect, the present disclosure provides an antibody or an antigen-binding fragment thereof, which is capable of specifically binding to TrkA and exhibits one or more properties selected from the group consisting of: 1) the antibody or the antigen-binding fragment thereof is a TrkA agonistic antibody or the antigen-binding fragment thereof, 2) being capable of binding with TrkA receptor and selectively activating the downstream signal pathway and avoiding p75 binding and its mediated biological effects; 3) being capable of promoting neuron growth and recovery in vitro and in vivo; 4) being capable of improving drug-like properties over NGF with longer half-life and/or less injection frequency; 5) not substantially blocking the binding between TrkA and NGF; 6) not substantially competing with NGF for binding to TrkA; 7) being capable of treating disease or disorder with less pain or irritation compared with NGF.

In some embodiments, the antibody is selected from the group consisting of: a monoclonal antibody, a chimeric antibody, a humanized antibody, a fully human antibody and a multi-specific antibody.

In some embodiments, the antigen-binding fragment is selected from the group consisting of: a Fab fragment, a Fab′ fragment, a F(ab)₂ fragment, a Fv fragment, a VHH and an ScFv.

In some embodiments, the antibody or the antigen-binding fragment is capable of competing with a reference antibody for binding to said TrkA, wherein said reference antibody comprises LCDR1-3 and HCDR1-3, said LCDR1 comprises the amino acid sequence as set forth in SEQ ID NO: 11, said LCDR2 comprises the amino acid sequence as set forth in SEQ ID NO: 12 (RT), said LCDR3 comprises the amino acid sequence as set forth in SEQ ID NO: 47, said HCDR1 comprises the amino acid sequence as set forth in SEQ ID NO: 43, said HCDR2 comprises the amino acid sequence as set forth in SEQ ID NO: 44, and said HCDR3 comprises the amino acid sequence as set forth in SEQ ID NO: 45.

In some embodiments, the antibody or the antigen-binding fragment comprises at least one of HCDR1-3 of a heavy chain variable region (VH), and said VH comprises an amino acid sequence as set forth in SEQ ID NO: 54.

In some embodiments, the antibody or the antigen-binding fragment comprises an HCDR3, and said HCDR3 comprises an amino acid sequence as set forth in SEQ ID NO: 45.

In some embodiments, the antibody or the antigen-binding fragment comprises an HCDR3, and said HCDR3 comprises an amino acid sequence as set forth in SEQ ID NO: 6 or SEQ ID NO: 16.

In some embodiments, the antibody or the antigen-binding fragment comprises an HCDR2, and said HCDR2 comprises an amino acid sequence as set forth in SEQ ID NO: 44.

In some embodiments, the antibody or the antigen-binding fragment comprises an HCDR2, and said HCDR2 comprises an amino acid sequence as set forth in any one of SEQ ID NO: 2, SEQ ID NO: 3, SEQ ID NO: 4 and SEQ ID NO: 5.

In some embodiments, the antibody or the antigen-binding fragment comprises an HCDR1, and said HCDR1 comprises an amino acid sequence as set forth in SEQ ID NO: 43.

In some embodiments, the antibody or the antigen-binding fragment comprises an HCDR1, and said HCDR1 comprises an amino acid sequence as set forth in SEQ ID NO: 1 or SEQ ID NO: 15.

In some embodiments, the antibody or the antigen-binding fragment comprises HCDR1, HCDR2 and HCDR3, said HCDR1 comprises an amino acid sequence as set forth in SEQ ID NO: 43, said HCDR2 comprises an amino acid sequence as set forth in SEQ ID NO: 44, and said HCDR3 comprises an amino acid sequence as set forth in SEQ ID NO: 45.

In some embodiments, the antibody or the antigen-binding fragment comprises a heavy chain variable region (VH), and said heavy chain variable region comprises an amino acid sequence as set forth in SEQ ID NO: 54.

In some embodiments, the antibody or the antigen-binding fragment comprises a heavy chain variable region (VH), and said heavy chain variable region comprises an amino acid sequence as set forth in any one of SEQ ID NO: 7, SEQ ID NO: 8, SEQ ID NO: 9, SEQ ID NO: 10, SEQ ID NO: 17, SEQ ID NO: 18, SEQ ID NO: 50 and SEQ ID NO: 52.

In some embodiments, the antibody or the antigen-binding fragment comprises a heavy chain constant region.

In some embodiments, the heavy chain constant region comprises a human IgG constant region.

In some embodiments, the antibody or the antigen-binding fragment comprises an LCDR3, and said LCDR3 comprises an amino acid sequence as set forth in SEQ ID NO: 47.

In some embodiments, the antibody or the antigen-binding fragment comprises an LCDR3, and said LCDR3 comprises an amino acid sequence as set forth in SEQ ID NO: 13 or SEQ ID NO: 19.

In some embodiments, the antibody or the antigen-binding fragment comprises an LCDR2, and said LCDR2 comprises an amino acid sequence as set forth in SEQ ID NO: 12 (RT).

In some embodiments, the antibody or the antigen-binding fragment comprises an LCDR1, and said LCDR1 comprises an amino acid sequence as set forth in SEQ ID NO: 11.

In some embodiments, the antibody or the antigen-binding fragment comprises LCDR1, LCDR2 and LCDR3, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 47.

In some embodiments, the antibody or the antigen-binding fragment comprises a light chain variable region (VL), and said light chain variable region comprises an amino acid sequence as set forth in SEQ ID NO: 55.

In some embodiments, the antibody or the antigen-binding fragment comprises a light chain variable region, and said light chain variable region comprises an amino acid sequence as set forth in any one of SEQ ID NO: 14, SEQ ID NO: 20, SEQ ID NO: 51 and SEQ ID NO: 53.

In some embodiments, the antibody or the antigen-binding fragment comprises a light chain constant region.

In some embodiments, the light chain constant region comprises a human Igκ constant region or a human Igλ constant region.

In some embodiments, the antibody or the antigen-binding fragment comprises HCDR1-3 and LCDR1-3, wherein the HCDR1-3 and LCDR1-3 comprises the amino acid sequences selected from the group consisting of:
1) said HCDR1 comprises an amino acid sequence as set forth in SEQ ID NO: 1, said HCDR2 comprises an amino acid sequence as set forth in SEQ ID NO: 2, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 13;
2) said HCDR1 comprises an amino acid sequence as set forth in SEQ ID NO: 1, said HCDR2 comprises an amino acid sequence as set forth in SEQ ID NO: 3, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 13;
3) said HCDR1 comprises an amino acid sequence as set forth in SEQ ID NO: 1, said HCDR2 comprises an amino acid sequence as set forth in SEQ ID NO: 4, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 13;
4) said HCDR1 comprises an amino acid sequence as set forth in SEQ ID NO: 1, said HCDR2 comprises an amino acid sequence as set forth in SEQ ID NO: 5, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 13;
5) said HCDR1 comprises an amino acid sequence as set forth in SEQ ID NO: 15, said HCDR2 comprises an amino acid sequence as set forth in SEQ ID NO: 3, said HCDR3 comprises an amino acid sequence as set forth in SEQ ID NO: 16, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 19;
6) said HCDR1 comprises an amino acid sequence as set forth in SEQ ID NO: 15, said HCDR2 comprises an amino acid sequence as set forth in SEQ ID NO: 5, said HCDR3 comprises an amino acid sequence as set forth in SEQ ID NO: 16, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 19; or
7) said HCDR1 comprises an amino acid sequence as set forth in SEQ ID NO: 15, said HCDR2 comprises an amino acid sequence as set forth in SEQ ID NO: 5, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 19.

In some embodiments, the antibody or the antigen-binding fragment comprises VH and VL, said VH and VL comprises amino acid sequences selected from the group consisting of:
1) said VH comprises an amino acid sequence as set forth in SEQ ID NO: 7, said VL comprises an amino acid sequence as set forth in SEQ ID NO: 14;
2) said VH comprises an amino acid sequence as set forth in SEQ ID NO: 8, said VL comprises an amino acid sequence as set forth in SEQ ID NO: 14;
3) said VH comprises an amino acid sequence as set forth in SEQ ID NO: 9, said VL comprises an amino acid sequence as set forth in SEQ ID NO: 14;
4) said VH comprises an amino acid sequence as set forth in SEQ ID NO: 10, said VL comprises an amino acid sequence as set forth in SEQ ID NO: 14;
5) said VH comprises an amino acid sequence as set forth in SEQ ID NO: 17, said VL comprises an amino acid sequence as set forth in SEQ ID NO: 20;
6) said VH comprises an amino acid sequence as set forth in SEQ ID NO: 18, said VL comprises an amino acid sequence as set forth in SEQ ID NO: 20;
7) said VH comprises an amino acid sequence as set forth in SEQ ID NO: 50, said VL comprises an amino acid sequence as set forth in SEQ ID NO: 51; or
8) said VH comprises an amino acid sequence as set forth in SEQ ID NO: 52, said VL comprises an amino acid sequence as set forth in SEQ ID NO: 53.

In another aspect, the present disclosure provides a fusion protein comprising the antibody or the antigen-binding fragment of the present disclosure.

In another aspect, the present disclosure provides a protein conjugate, comprising the antibody or the antigen-binding fragment of the present disclosure, or the fusion protein of the present disclosure.

In another aspect, the present disclosure provides one or more isolated nucleic acid molecule or molecules, encoding for the antibody or the antigen-binding fragment of the present disclosure, or the fusion protein of the present disclosure.

In another aspect, the present disclosure provides one or more vector or vectors, comprising the one or more isolated nucleic acid molecule or molecules of the present disclosure.

In another aspect, the present disclosure provides a cell, comprising the isolated nucleic acid molecule or molecules or the vector or vectors of the present disclosure.

In another aspect, the present disclosure provides a composition, comprising the antibody or the antigen-binding fragment, the fusion protein, the protein conjugate, the isolated nucleic acid molecule or molecules, the vector or vectors, and/or the cell of the present disclosure, and optionally a pharmaceutically acceptable excipient.

In some embodiments, the pharmaceutically acceptable excipient in the composition comprises a buffer.

In another aspect, the present disclosure provides use of the antibody or the antigen-binding fragment thereof, the fusion protein, the protein conjugate, the isolated nucleic acid molecule or molecules, the vector or vectors, the cell, and/or the composition of the present disclosure in the manufacture of a medicament for preventing and/or treating a disease or disorder.

In some embodiments, the disease or disorder comprise tumors, peripheral neuropathies, ophthalmic diseases, diabetes and related complications, cardiovascular disease, and/or Alzheimer's disease.

In some embodiments, the tumors comprise neuronal tumors or non-neuronal tumors.

In some embodiments, the tumors comprise neuroblastoma, glioblastoma, medulloblastoma, prostatic cancer, epithelial ovarian cancer, non-small cell lung cancer, and/or breast cancer.

In some embodiments, the peripheral neuropathies comprise chemotherapy-induced peripheral neuropathy (CIPN), and/or diabetic neuropathy.

In some embodiments, the ophthalmic diseases comprises dry eye disease (DED).

In another aspect, the present disclosure provides a method for preventing and/or treating a disease and/or disorder in a subject in need thereof, comprising administering to the subject an effective amount of the antibody or the antigen-binding fragment the fusion protein, the protein conjugate, the isolated nucleic acid molecule or molecules, the vector or vectors, the cell, and/or the composition of the present disclosure.

In some embodiments, the disease or disorder comprise tumors, peripheral neuropathies, ophthalmic diseases, diabetes and related complications, cardiovascular disease, and/or Alzheimer's disease.

In some embodiments, the tumors comprise neuronal tumors or non-neuronal tumors.

In some embodiments, the tumors comprise neuroblastoma, glioblastoma, medulloblastoma, prostatic cancer, epithelial ovarian cancer, non-small cell lung cancer, and/or breast cancer.

In some embodiments, the peripheral neuropathies comprise chemotherapy-induced peripheral neuropathy (CIPN), and/or diabetic neuropathy.

In some embodiments, the ophthalmic diseases comprises dry eye disease (DED).

In another aspect, the present disclosure provides the antibody or the antigen-binding fragment thereof, the fusion protein, the protein conjugate, the isolated nucleic acid molecule or molecules, the vector or vectors, the cell, or the composition of the present disclosure for use in preventing and/or treating a disorder or disease.

In some embodiments, the disease or disorder comprise tumors, peripheral neuropathies, ophthalmic diseases, diabetes and related complications, cardiovascular disease, and/or Alzheimer's disease.

In some embodiments, the tumors comprise neuronal tumors or non-neuronal tumors.

In some embodiments, the tumors comprise neuroblastoma, glioblastoma, medulloblastoma, prostatic cancer, epithelial ovarian cancer, non-small cell lung cancer, and/or breast cancer.

In some embodiments, the peripheral neuropathies comprise chemotherapy-induced peripheral neuropathy (CIPN), and/or diabetic neuropathy.

In some embodiments, the ophthalmic diseases comprises dry eye disease (DED).

In another aspect, the present disclosure provides a method for producing the antibody or the antigen-binding fragment thereof, or the fusion protein of the present disclosure, comprising culturing the cell of the present disclosure under conditions enabling expression of the antibody or the antigen-binding fragment, or the fusion protein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates the TrkA activation dependent NFAT promoter driven reporter assay for selected hybridoma antibodies.

FIG. 2 illustrates the TrkA activation dependent Akt phosphorylation detected by HTRF assay for selected hybridoma antibodies.

FIG. 3 illustrates the TrkA antibodies FACS binding on TrkA-NFAT-bla CHO-K1 cells (Chi70D3A6 and Hu70D3A6-V12).

FIG. 4 illustrates the TrkA antibodies FACS binding on TrkA-NFAT-bla CHO-K1 cells (Chi99H4D5 and Hu99H4D5-V6).

FIG. 5 illustrates the TrkA activation dependent NFAT promoter driven reporter assay for selected chimeric antibodies (chi99H4D5, chi70D3A6).

FIG. 6 illustrates the TrkA activation dependent Akt phosphorylation detected by HTRF assay for selected chimeric antibodies (chi99H4D5, chi70D3A6).

FIG. 7 illustrates the TrkA activation dependent Akt phosphorylation detected by HTRF assay for selected humanized antibodies (Hu99H4D5-V6 and Hu70D3A6-V12).

FIG. 8 illustrates the PK study in male SD rat.

FIG. 9 illustrates the NGF block assay result.

FIG. 10 illustrates the Brightfield axon number and neurite lenght statistics after 72 hours of DRG seeding (using chi99H4D5, chi70D3A6).

FIG. 11 illustrates the Brightfield neurite lenght statistics after 72 hours of DRG seeding (using Hu99H4D5-V6, Hu70D3A6-V12).

FIG. 12 illustrates the TrkA agonist antibodies (chi99H4D5, chi70D3A6) do not elicit mechanical hyperalgesia in vivo.

FIG. 13 illustrates the TrkA agonist antibodies (Hu99H4D5-V6, Hu70D3A6-V12) do not elicit mechanical hyperalgesia in vivo.

FIG. 14 illustrates neurotrophic effect of the TrkA agonist antibodies (Hu99H4D5-V6, Hu70D3A6-V12) in vivo.

### DETAILED DESCRIPTION

The term “antibody”, as used herein, generally refers to an immunoglobulin or an immunoglobulin-like molecule capable of specifically recognizing or binding to an antigen. An antibody may comprise a light chain (L) and a heavy chain (H). The light chains of an antibody can be classified as κ and λ light chains. The heavy chains can be classified as μ, δ, γ, α or ε, and the isotypes of an antibody are defined as IgM, IgD, IgG (e.g., IgG1, IgG2, IgG3 or IgG4 subtype), IgA and IgE, respectively. Each heavy chain may comprise a heavy chain variable region (VH) and a heavy chain constant region (CH). The heavy chain constant region may comprise three domains (CH1, CH2 and CH3). Each light chain may comprise a light chain variable region (VL) and a light chain constant region (CL). The light chain constant region may comprise a CL domain. The VH and VL regions can also be subdivided into regions with high variability known as complementarity determining regions (CDRs) interspersed with more conserved regions known as framework regions (FRs). Each VH and VL consists of 3 CDRs and 4 FRs arranged from N-terminal to C-terminal in the following order: FR1, CDR1, FR2, CDR2, FR3, CDR3, FR4. The variable regions (VH and VL) of each heavy/light chain pair form the antibody binding site, respectively. Distribution of amino acids to regions or domains follows the definition of Kabat Sequences of Proteins of Immunological Interest (National Institutes of Health, Bethesda, Md. (1987 and 1991)), or Chothia & Lesk (1987) J. Mol. Biol. 196:901-917; Chothia et al. (1989) Nature 342:878-883. The term “antibody” is not limited by any antibody-producing method. For example, it includes recombinant antibodies, monoclonal antibodies, and other forms of antibodies. In some cases, an antibody of the present disclosure is an isolated antibody.

The term “antigen-binding fragment”, as used herein, generally refers to one or more fragments of a full-length antibody that retain the ability to bind the same antigen to which the antibody binds (e.g., TrkA) and/or competes against an intact antibody for an antigen-specific binding. Antigen-binding fragment can be produced by recombinant DNA techniques or by enzymatic or chemical cleavage of intact antibodies. In some cases, the antigen-binding fragment includes Fab, Fab′, F (ab′)₂, F(ab)₂, VHH, Fd, Fv, dAb and complementarity determining region (CDR) fragments, single chain antibodies (e.g., ScFv), chimeric antibodies, diabodies, and polypeptides that comprise at least a portion of an antibody that is sufficient to confer specific antigen-binding ability to the polypeptide.

The term “TrkA”, as used herein, generally refers to high affinity nerve growth factor receptor or neurotrophic tyrosine kinase receptor type 1 or TRK1-transforming tyrosine kinase protein or Tropomyosin-related kinase A or Tyrosine kinase receptor or Tyrosine kinase receptor A or Trk-A or gp140trk or p140-TrkA or MTC or TRK. TrkA is a receptor tyrosine kinase involved in the development and the maturation of the central and peripheral nervous systems through regulation of proliferation, differentiation, and survival of sympathetic and nervous neurons. TrkA is the high affinity receptor for NGF which is its primary ligand; it may also bind and be activated by NTF3/neurotrophin-3. TrkA as used herein may comprise functional fragments, variants, isoforms, and species homologs of human TrkA. Accordingly, antibodies of this disclosure may, in certain cases, cross-react with TrkA from species other than human. In certain embodiments, the antibodies may be completely specific for one or more human TrkA proteins and may not exhibit species or other types of non-human cross-reactivity. The complete amino acid sequence of the four known human TrkA isoforms are found under the UniProt/Swiss-Prot accession number P04629 (Consortium TU, (2012) Nucleic Acids Res. 40(D1):D71-D5). The four isoforms are produced by alternative splicing: isoform TrkA-I is found in most non-neuronal tissues (UniProt/Swiss-Prot accession number P04629-2), while isoform TrkA-II is primarily expressed in neuronal cells (UniProt/Swiss-Prot accession number P04629-1), and isoform TrkA-III is specifically expressed by pluripotent neural stem and neural crest progenitors (UniProt/Swiss-Prot accession number P04629-4). A fourth isoform which differs from isoform TrkA-II at residues 1-71 and lacks residues 393 to 398 is known as isoform 3 (UniProt/Swiss-Prot accession number P04629-3). TrkA-II isoform is the major known isoform of TrkA. Isoform TrkA-I has enhanced responsiveness to NTF3 neurotrophin whereas isoform TrkA-III is constitutively active and does not bind NGF.

The term “TrkA/NGF signaling pathway”, as used herein, generally refers to the signaling pathway relating to Nerve Growth Factor (NGF) and Tyrosine Kinase A (TrkA). The NGF peptides may engage the NGF pathway through TrkA phosphorylation, and activation of ShcC/PI3K and Plc-7/MAPK signaling, promoting AKT-dependent survival and CREB-driven neuronal activity. The TrkA/NGF signaling pathway may promote survival and innervation of sympathetic and sensory neurons, for example, may relate to the endocytosis and retrograde transport of NGF/TrkA-containing endosomes from the axon terminal to the cell body for activation of NGF-inducible gene expression responsible for neuronal survival and development.

The term “monoclonal antibody”, as used herein, generally refers to antibodies that are made by identical immune cells that are all clones of a unique parent cell. Monoclonal antibodies can have a monovalent affinity, in that they bind to the same epitope (the part of an antigen that is recognized by the antibody). Sometimes, monoclonal antibodies may also be multi specific, such as bispecific or tri specific. It has become an important tool in biochemistry, molecular biology, and medicine. Several monoclonal antibody technologies had been developed recently, such as phage display, single B cell culture, single cell amplification from various B cell populations and single plasma cell interrogation technologies.

The term “chimeric antibody”, as used herein, generally refers to an antibody in which the Variable (V) region of light and heavy chains is of mouse origin, while the Constant (C) region is of human origin. In general, the chimeric antibody may retain the specificity and affinity of the original mouse monoclonal antibody, but HAMA response may be significantly reduced.

The term “humanized antibody”, as used herein, generally refers to antibodies from non-human species whose protein sequences have been modified to increase their similarity to antibody variants produced naturally in humans. The amino acid sequence of a humanized antibody may be essentially identical to that of a human variant, despite the non-human origin of some of its complementarity determining region (CDR) segments responsible for the ability of the antibody to bind to its target antigen.

The term “fully human antibody” and “human antibody” are used interchangeably herein, and generally refers to an antibody that comprises a human variable region and, most preferably a human constant region. In specific embodiments, the terms refer to an antibody that comprises a variable region and constant region of human origin. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). The term “Fab fragment”, as used herein, generally refers to a portion (such as an antigen-binding fragment) of an immunoglobulin molecule. A Fab fragment may comprise one light chain and part of a heavy chain with a single antigen-binding site. A Fab fragment may be obtained by papain digestion of an immunoglobulin molecule. For example, a Fab fragment may be composed of one constant and one variable domain of each of the heavy and the light chain. The variable domain may contain the paratope (the antigen-binding site), comprising a set of the complementarity determining regions, at the amino-terminal end of the immunoglobulin molecule. The enzyme papain may be used to cleave an immunoglobulin molecule into two Fab fragments and one Fc fragment. The enzyme pepsin cleaves below the hinge region, so a F(ab′)₂ fragment and a pFc′ fragment is formed. Divalent F(ab)₂ or F(ab′)₂ fragments have two antigen-binding regions that are linked by disulfide bonds. Reduction of F(ab)₂ or F(ab′)₂ fragments produce 2 monovalent Fab or Fab′ fragments, which have a free sulfhydryl group that is useful for conjugation to other molecules.

The term “Fv fragment”, as used herein, generally refers to the smallest fragment made from enzymatic cleavage of IgG and IgM class antibodies. Fv fragments have the antigen-binding site made of the VH and VL regions, but they lack the CH1 and CL regions. The VH and VL chains may be held together in Fv fragments by non-covalent interactions.

The term “ScFv”, as used herein, generally refers to a single-chain antibody fragment. An ScFv may be a recombinant single chain polypeptide molecule in which light and heavy chain variable regions of an antibody are connected, either directly or via a peptide linker. Single chain antibodies (ScFv) generally do not include portions of the Fc region of antibody, although methods are known for adding such regions to known ScFv molecules if desired. See Helfrich et al., A rapid and versatile method for harnessing ScFv antibody fragments with various biological functions. J Immunol Methods 237:131-145 (2000) and de Haard et al., Creating and engineering human antibodies for immunotherapy. Advanced Drug Delivery Reviews 31:5-31 (1998).

The term “fusion protein”, as used herein, generally refers to a polypeptide that comprises, or alternatively consists of, an amino acid sequence of a polypeptide fused directly or indirectly (e.g., via a linker) to an amino acid sequence of a heterologous polypeptide (i.e., a polypeptide of a different origin, sequence or structure).

The term “protein conjugate”, as used herein, generally refers to a conjugate comprising a protein (e.g., an antibody or a functional fragment thereof) conjugated to one or more additional moieties, such as cytotoxic agents, e.g., a chemotherapeutic agent, a drug, a growth inhibitory agent, a toxin (e.g., a protein toxin, an enzymatically active toxin of bacterial, fungal, plant, or animal origin, or fragments thereof), a label (e.g., a fluorescent label) and/or a radioactive isotope (i.e., a radio-conjugate).

A number of CDR definitions are in use and are encompassed herein. The Kabat definition is based on sequence variability and is the most commonly used (Kabat E A et al., ibid.). Chothia definition is based on the location of the structural loops (Chothia & Lesk J. (1987) Mol. Biol. 196:901-917). The AbM definition is a compromise between the Kabat and the Chothia definitions and is used by Oxford Molecular's AbM antibody modelling software (Martin A C R et al., (1989) PNAS USA 86:9268-9272; Martin A C R et al., (1991) Methods Enzymol. 203:121-153; Pedersen J T et al., (1992) Immunomethods 1:126-136; Rees A R et al., (1996) In Sternberg M. J. E. (ed.), Protein Structure Prediction. Oxford University Press, Oxford, 141-172). The contact definition has been recently introduced (MacCallum R M et al., (1996) J. Mol. Biol. 262:732-745) and is based on an analysis of the available complex structures available in the Protein Databank. The definition of the CDR by IMGT®, the international ImMunoGeneTics information System® (http://www.imgt.org) is based on the IMGT numbering for all immunoglobulin and T cell receptor V-REGIONs of all species (IMGT®, the international ImMunoGeneTics information System®; Lefranc M P et al., (1999) Nucleic Acids Res. 27(1):209-12; Ruiz M et al., (2000) Nucleic Acids Res. 28(1):219-21; Lefranc M P (2001) Nucleic Acids Res. 29(1):207-9; Lefranc M P (2003) Nucleic Acids Res. 31(1):307-10; Lefranc M P et al., (2005) Dev. Comp. Immunol. 29(3):185-203; Kaas Q et al., (2007) Briefings in Functional Genomics & Proteomics, 6(4):253-64).

The term “isolated nucleic acid molecule or molecules” as used herein, generally refers to a polymeric form of nucleotides of any length, either deoxyribonucleotides or ribonucleotides, or analogs thereof, isolated from its native environment, or that is artificially synthesized.

The term “vector or vectors” as used herein, generally refers to a nucleic acid vehicle into which a polynucleotide encoding a protein can be inserted and expressed. The genetic material elements carried in the vector can be expressed in a host cell by transforming, transducing, or transfecting the host cell with the vector. A vector may contain a variety of elements that control expression, including promoter sequences, transcriptional initiation sequences, enhancer sequences, selection elements, and reporter genes. In addition, the vector may also contain an origin of replication. It is also possible that the vector may include components that assist its entry into the cell, such as viral particles, liposomes or protein shells, but not only these substances.

The term “cell” as used herein, generally refers to a cell that may be used to carry the vector or vectors of the present disclosure, or be used to express or produce the antibody, the antigen-binding fragment of the present disclosure. A cell of the present disclosure may be a host cell.

The terms “disease” and “disorder” may be used interchangeably herein, and generally refer to any condition that impairs the normal functioning of the body. Disease is often construed as a medical condition associated with specific symptoms and signs. It may be caused by external factors such as pathogens or by internal dysfunctions.

As used herein, the term “subject” includes any human or non-human animal. The term “non-human animal” includes all vertebrates, e.g., mammals and non-mammals, such as primates, sheep, dogs, cats, horses, cows, chickens, amphibians, reptiles, etc. For example, the subject may be human.

The term “effective amount”, as used herein, generally refers to a dose sufficient to provide concentrations high enough to impart a beneficial effect on the recipient thereof. The specific therapeutically effective dose level for any particular subject will depend upon a variety of factors including the disease or disorder being treated, the severity of the disease or disorder, the activity of the specific component, the route of administration, the rate of clearance, the duration of treatment, the age, body weight, sex, diet, and general health of the subject, and other related factors.

The term “pharmaceutically acceptable excipient”, as used herein, generally refers to any and all solvents, dispersion media, coatings, isotonic and absorption delaying agents, etc., that are compatible with pharmaceutical administration.

The term “about”, as used herein, generally refers to an approximation to a given value that would reasonably be inferred based on the ordinary skill in the art, including equivalents and approximations due to the experimental and/or measurement conditions for such given value. For example, it may refer to a value that is no more than 10% above or below the value being modified by the term.

The terms “polypeptide” or “protein”, as used herein, generally refers to macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally occurring amino acid and polymers. The terms “polypeptide” and “protein” specifically encompass TrkA antigen-binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 1 10, 150, 200, 250, 300, 350, 400, or 450 amino acids long. Useful polypeptide fragments include immunologically functional fragments of antibodies, including binding domains. In the case of a TrkA-binding antibody, useful fragments include but are not limited to a CDR region, a variable domain of a heavy and/or light chain, a portion of an antibody chain or just its variable region including two CDRs, and the like.

The term “isolated protein” (such as isolated antibody), as used herein, generally refers to a subject protein (1) is free of at least some other proteins with which it would normally be found, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is operably associated (by covalent or noncovalent interaction) with a polypeptide with which it is not associated in nature, or (6) does not occur in nature. Typically, an “isolated protein” constitutes at least about 5%, at least about 10%, at least about 25%, or at least about 50% of a given sample. Genomic DNA, cDNA, mRNA or other RNA, of synthetic origin, or any combination thereof can encode such an isolated protein. Preferably, the isolated protein is substantially free from proteins or polypeptides or other contaminants that are found in its natural environment that would interfere with its therapeutic, diagnostic, prophylactic, research or other use.

A “variant” of a polypeptide (e.g., an antigen-binding protein, or an antibody) comprises an amino acid sequence wherein one or more amino acid residues are inserted into, deleted from and/or substituted into the amino acid sequence relative to another polypeptide sequence. Variants include fusion proteins.

Conservative amino acid substitutions can encompass non-naturally occurring amino acid residues, which are typically incorporated by chemical peptide synthesis rather than by synthesis in biological systems. These include peptidomimetics and other reversed or inverted forms of amino acid moieties.

An “Fc” region comprises two heavy chain fragments comprising the C_{H1} and C_{H2} domains of an antibody. The two heavy chain fragments are held together by two or more disulfide bonds and by hydrophobic interactions of the C_{H3} domains.

A “multi-specific antigen-binding protein” or “multi-specific antibody” is one that targets more than one antigen or epitope.

A “bispecific”, “dual-specific” or “bifunctional” antigen-binding protein or antibody is a hybrid antigen-binding protein or antibody, respectively, having two different antigen-binding sites. Bispecific antigen-binding proteins and antibodies are a species of multispecific antigen-binding protein antibody and can be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al, 1992, J. Immunol. 148:1547-1553. The two binding sites of a bispecific antigen-binding protein or antibody will bind to two different epitopes, which can reside on the same or different protein targets.

Antigen-binding region” or “antigen-binding fragment” means a protein, or a portion of a protein, that specifically binds a specified antigen (e.g., a paratope). For example, that portion of an antigen-binding protein that contains the amino acid residues that interact with an antigen and confer on the antigen-binding protein its specificity and affinity for the antigen is referred to as “antigen-binding region”. An antigen-binding region typically includes one or more “complementary binding regions” (“CDRs”). A “CDR” is an amino acid sequence that contributes to antigen-binding specificity and affinity.

Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the IMGT definition, the AbM definition and the contact definition.

The term “compete”, when used in the context of antigen-binding proteins (e.g., antigen-binding proteins or antibodies) that compete for the same epitope means competition between antigen-binding proteins as determined by an assay in which the antigen-binding protein (e.g., antibody or immunologically functional fragment thereof) being tested prevents or inhibits (e.g., reduces) specific binding of a reference antigen-binding protein (e.g., a ligand, or a reference antibody) to a common antigen (e.g., TrkA or a fragment thereof, such as the ECD thereof). Numerous types of competitive binding assays can be used to determine if one antigen-binding protein competes with another, for example: solid phase direct or indirect radioimmunoassay (RIA), solid phase direct or indirect enzyme immunoassay (EIA), sandwich competition assay (see, e.g., Stahli et al, 1983, Methods in Enzymology 9:242-253); solid phase direct biotin-avidin EIA (see, e.g., Kirkland et al, 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al, 1988, Molec. Immunol. 25:7-15); solid phase direct biotin-avidin EIA (see, e.g., Cheung, et al, 1990, Virology 176:546-552); and direct labeled RIA (Moldenhauer et al, 1990, Scand. J. Immunol. 32:77-82). Typically, such an assay involves the use of purified antigen bound to a solid surface or cells bearing either of these, an unlabeled test antigen-binding protein and a labeled reference antigen-binding protein. Competitive inhibition is measured by determining the amount of label bound to the solid surface or cells in the presence of the test antigen-binding protein. Usually, the test antigen-binding protein is present in excess. Antigen-binding proteins identified by competition assay (competing antigen-binding proteins) include antigen-binding proteins binding to the same epitope as the reference antigen-binding proteins and antigen-binding proteins binding to an adjacent epitope sufficiently proximal to the epitope bound by the reference antigen-binding protein for steric hindrance to occur. Usually, when a competing antigen-binding protein is present in excess, it will inhibit (e.g., reduce) specific binding of a reference antigen-binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

The term “antigen”, as used herein, generally refers to a molecule or a portion of a molecule capable of being bound by a selective binding agent, such as an antigen-binding protein (including, e.g., an antibody or immunological functional fragment thereof). In some embodiments, the antigen is capable of being used in an animal to produce antibodies capable of binding to that antigen. An antigen can possess one or more epitopes that are capable of interacting with different antigen-binding proteins, e.g., antibodies.

The term “epitope” includes any determinant capable of being bound by an antigen-binding protein, such as an antibody or to a T-cell receptor. An epitope is a region of an antigen that is bound by an antigen-binding protein that targets that antigen, and when the antigen is a protein, includes specific amino acids that directly contact the antigen-binding protein. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially recognize an epitope on the target antigen in a complex mixture of proteins and/or macromolecules.

As used herein, “substantially” or “substantial” generally means to a great or significant extent (e.g., to an extent of at least 50%, at least 60%, at least 70%, at least 80%, at least 85%, at least 90%, at least 95%, or at least 99% or more).

The term “agent” is used herein to denote a chemical compound, a mixture of chemical compounds, a biological macromolecule, or an extract made from biological materials.

The term “treat” and “treatment” includes therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors.

The term “prevent” does not require the 100% elimination of the possibility of an event. Rather, it denotes that the likelihood of the occurrence of the event has been reduced in the presence of the compound or method.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al, Molecular Cloning: A Laboratory Manual (2d ed., Cold Spring Harbor Laboratory Press, Cold Spring Harbor, N. Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients.

### Antibody or an Antigen-Binding Fragment Thereof

In one aspect, the present disclosure provides an antibody or an antigen-binding fragment thereof, which binds to TrkA.

The antibody or the antigen-binding fragment thereof may specifically bind to TrkA. For example, the antibody or the antigen-binding fragment may bind to TrkA with a K_{D} of less than about 1.0×10⁻⁸M, as measured by Octet or SPR. For example, with a K_{D} of less than about 1×10⁻⁹M, less than about 1.3×10⁻⁹M, less than about 2×10⁻⁹M, less than about 3×10⁻⁹M, less than about 4×10⁻⁹M, less than about 5×10⁻⁹M, less than about 6×10⁻⁹M, less than about 7×10⁻⁹M, less than about 9×10⁻⁹M, less than about 1×10⁻¹⁰ M, less than about 7×10⁻¹⁰ M, less than about 1×10⁻⁹ M, or less than about 1×10⁻²M, or a K_{D} value that is even smaller.

The antibody or the antigen-binding fragment thereof of the present disclosure may be a TrkA agonistic antibody or the antigen-binding fragment thereof.

The antibody or the antigen-binding fragment thereof may be capable of binding with TrkA receptor and selectively activating the downstream signal pathway and avoiding p75 binding and its mediated biological effects.

The antibody or the antigen-binding fragment thereof may be capable of promoting neuron growth and recovery in vitro and in vivo.

The antibody or the antigen-binding fragment thereof may be capable of improving drug-like properties over NGF with longer half-life and/or less injection frequency.

The antibody or the antigen-binding fragment thereof may not block the binding between TrkA and NGF.

The antibody, or the antigen-binding fragment thereof may be capable of treating disease or disorder with less pain or irritation compared with NGF.

TrkA also known as high affinity nerve growth factor receptor, neurotrophic tyrosine kinase receptor type 1, or TRK1-transforming tyrosine kinase protein is a protein that is encoded by the NTRKI gene. In the present disclosure, the TrkA may be a human TrkA or TrkA derived from any other species. An exemplary human TrkA amino acid sequence is as set forth in SEQ ID NO: 41. As described herein, TrkA proteins can also include fragments of the full length TrkA protein, such as the extracellular domain (ECD) thereof. An exemplary human TrkA ECD amino acid sequence is as set forth in SEQ ID NO: 42.

In some embodiments, the antibody, or the antigen-binding fragment thereof comprises one or more CDRs (e.g., 1, 2, 3, 4, 5 or 6 CDRs). In some embodiments, the antibody, or the antigen-binding fragment thereof comprises (a) a polypeptide structure and (b) one or more CDRs that are inserted into and/or joined to the polypeptide structure. The polypeptide structure can take a variety of different forms. For example, it can be, or comprise, the framework of a naturally occurring antibody, or fragment or variant thereof, or can be completely synthetic in nature.

In certain embodiments, the polypeptide structure of the antibody, or the antigen-binding fragment thereof is an antibody or is derived from an antibody, including, but not limited to, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and portions or fragments of each, respectively. In some instances, the antibody, or the antigen-binding fragment thereof is an immunological fragment of an antibody (e.g., a Fab, a Fab′, a F(ab′)₂, or a scFv).

The antibody, or the antigen-binding fragment thereof may comprise a heavy chain constant region. The heavy chain constant region may comprise a human IgG constant region (such as a human IgG1, IgG2, or IgG4 constant region). In some embodiments, the heavy chain constant region comprises a human IgG4 constant region.

The antibody, or the antigen-binding fragment thereof may comprise a light chain constant region. The light chain constant region may comprise a human Igκ constant region or a human Igλ constant region. In some embodiments, the light chain constant region may comprise a human Igκ constant region.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain/light chain pair mentioned above typically are aligned by the framework regions to form a structure that binds specifically with a specific epitope on the target protein (e.g., TrkA). From N-terminal to C-terminal, naturally occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in IMGT numbering system.

Various heavy chain and light chain variable regions are provided herein. In some embodiments, each of these variable regions can be attached to a heavy and light chain constant region to form a complete antibody heavy and light chain, respectively. Further, each of the so generated heavy and light chain sequences can be combined to form a complete antibody structure.

Specific examples of some of the variable regions of the light (VL) and heavy (VH) chains of the antibodies are provided and their corresponding amino acid sequences are summarized in Table 1 below.

TABLE 1

| Antibody | VH | VL |
|---|---|---|
| General formula | SEQ ID NO: 46 | SEQ ID NO: 48 |
| Chi99H4D5 | SEQ ID NO: 7 | SEQ ID NO: 14 |
| Chi99H4D5-V1 | SEQ ID NO: 8 | SEQ ID NO: 14 |
| Chi99H4D5-V2 | SEQ ID NO: 9 | SEQ ID NO: 14 |
| Chi99H4D5-V3 | SEQ ID NO: 10 | SEQ ID NO: 14 |
| Hu99H4D5-V6 | SEQ ID NO: 52 | SEQ ID NO: 53 |
| Chi70D3A6 | SEQ ID NO: 17 | SEQ ID NO: 20 |
| Chi70D3A6-V1 | SEQ ID NO: 18 | SEQ ID NO: 20 |
| Hu70D3A6-V12 | SEQ ID NO: 50 | SEQ ID NO: 51 |

Each of the exemplary variable heavy chains listed in Table 1 can be combined with any of the exemplary variable light chains shown in Table 1 to form an antibody. Table 1 shows exemplary light and heavy chain variable region pairings found in several of the antibodies disclosed herein. In some instances, the antibodies include at least one variable heavy chain and one variable light chain from those listed in Table 1. In other instances, the antibodies contain two identical light chains and two identical heavy chains. As an example, an antibody or antigen-binding fragment thereof can include a heavy chain and a light chain, two heavy chains, or two light chains. In some embodiments, the antigen-binding fragment thereof comprises (and/or consists of) 1, 2, and/or 3 heavy and/or light CDRs from at least one of the sequences listed in Table 1. In some embodiments, all 6 CDRs (CDR1-3 from the light (LCDR1, LCDR2, LCDR3) and CDR 1-3 from the heavy (HCDR1, HCDR2, and HCDR3)) are part of the antibody or the antigen-binding fragment thereof. In some embodiments, 1, 2, 3, 4, 5, or more CDRs are included in the antibody or the antigen-binding fragment thereof. In some embodiments, one heavy and one light CDR from the CDRs in the sequences in Table 1 is included in the antibody or the antigen-binding fragment thereof. In some embodiments, additional sections are also included in the antibody or the antigen-binding fragment thereof. Optional light chain variable sequences (including CDR1, CDR2, and CDR3) can be selected from the following: SEQ ID NO: 14, SEQ ID NO: 20, SEQ ID NO: 51 or SEQ ID NO: 53. Optional heavy chain variable sequences (including CDR1, CDR2 and CDR3) can be selected from the following: SEQ ID NO: 7, SEQ ID NO: 8, SEQ ID NO: 9, SEQ ID NO: 10, SEQ ID NO: 17, SEQ ID NO: 18, SEQ ID NO: 50 or SEQ ID NO: 52.

In some embodiments, the antibody or the antigen-binding fragment thereof comprises (and/or consists of) LCDR1 and LCDR3 from at least one of the sequences listed in Table 1.

In some embodiments, the antibody or the antigen-binding fragment thereof comprises (and/or consists of) HCDR1 and HCDR3 from at least one of the sequences listed in Table 1.

In some embodiments, the antibody or the antigen-binding fragment thereof comprises (and/or consists of) LCDR1, LCDR3, HCDR1 and HCDR3 from at least one of the sequences listed in Table 1.

Examples of the CDRs of the antibodies shown in Table 1 are listed below (determined according to the IMGT method) in Table 2.

TABLE 2
(IMGT)

| Antibody | HCDR1 | HCDR2 | HCDR3 | LCDR1 | LCDR2 | LCDR3 |
|---|---|---|---|---|---|---|
| General | SEQ ID | SEQ ID | SEQ ID | SEQ ID | SEQ ID NO: | SEQ ID |
| formula | NO: 43 | NO: 44 | NO: 45 | NO: 11 | 12 (RT) | NO: 47 |
| Chi99H4D5 | SEQ ID | SEQ ID | SEQ ID | SEQ ID | SEQ ID NO: | SEQ ID |
| | NO: 1 | NO: 2 | NO: 6 | NO: 11 | 12 (RT) | NO: 13 |
| Chi99H4D5- | SEQ ID | SEQ ID | SEQ ID | SEQ ID | SEQ ID NO: | SEQ ID |
| V1 | NO: 1 | NO: 3 | NO: 6 | NO: 11 | 12 (RT) | NO: 13 |
| Chi99H4D5- | SEQ ID | SEQ ID | SEQ ID | SEQ ID | SEQ ID NO: | SEQ ID |
| V2 | NO: 1 | NO: 4 | NO: 6 | NO: 11 | 12 (RT) | NO: 13 |
| Chi99H4D5- | SEQ ID | SEQ ID | SEQ ID | SEQ ID | SEQ ID NO: | SEQ ID |
| V3 | NO: 1 | NO: 5 | NO: 6 | NO: 11 | 12 (RT) | NO: 13 |
| Hu99H4D5- | SEQ ID | SEQ ID | SEQ ID | SEQ ID | SEQ ID NO: | SEQ ID |
| V6 | NO: 1 | NO: 5 | NO: 6 | NO: 11 | 12 (RT) | NO: 13 |
| Chi70D3A6 | SEQ ID | SEQ ID | SEQ ID | SEQ ID | SEQ ID NO: | SEQ ID |
| | NO: 15 | NO: 3 | NO: 16 | NO: 11 | 12 (RT) | NO: 19 |
| Chi70D3A6- | SEQ ID | SEQ ID | SEQ ID | SEQ ID | SEQ ID NO: | SEQ ID |
| V1 | NO: 15 | NO: 5 | NO: 16 | NO: 11 | 12 (RT) | NO: 19 |
| Hu70D3A6- | SEQ ID | SEQ ID | SEQ ID | SEQ ID | SEQ ID NO: | SEQ ID |
| V12 | NO: 15 | NO: 5 | NO: 6 | NO: 11 | 12 (RT) | NO: 19 |

Some of the exemplary antibodies provided in the present disclosure are considered alternatives or variants to each other.

In some embodiments, the antibody or the antigen-binding fragment thereof is capable of binding to an epitope recognized by one of the antibodies described in Table 1. In some embodiments, the antibody, or the antigen-binding fragment thereof is capable of binding to a specific conformational state of TrkA so as to activate TrkA signaling pathway while not blocking the binding of TrkA with NGF.

As described herein, the TrkA antibody or the antigen-binding fragment thereof can comprise a humanized antibody and/or part thereof. An important practical application of such a strategy is the “humanization” of the mouse humoral immune system. In certain embodiments, a humanized antibody is substantially non-immunogenic in humans. In certain embodiments, a humanized antibody has substantially the same affinity for a target as an antibody from another species from which the humanized antibody is derived.

In certain embodiments, amino acids of an antibody variable domain that can be modified without diminishing the native affinity of the antigen-binding domain while reducing its immunogenicity are identified.

In certain embodiments, modification of an antibody by methods known in the art is typically designed to achieve increased binding affinity for a target and/or to reduce immunogenicity of the antibody in the recipient. In certain embodiments, humanized antibodies are modified to eliminate glycosylation sites in order to increase affinity of the antibody for its cognate antigen. See, e.g., Co et al., MoI. Immunol., 30:1361-1367 (1993). In certain embodiments, techniques such as “reshaping”, “hyperchimerization” or “veneering/resurfacing” are used to produce humanized antibodies. See, e.g., Vaswami et al., Annals of Allergy, Asthma, & Immunol. 81:105 (1998); Roguska et al, Prot. Engineer., 9:895-904 (1996); and U.S. Pat. No. 6,072,035. In certain such embodiments, such techniques typically reduce antibody immunogenicity by reducing the number of foreign residues, but do not prevent anti-idiotypic and anti-allotypic responses following repeated administration of the antibodies.

In certain instances, humanizing antibodies results in a loss of antigen-binding capacity. In certain embodiments, humanized antibodies are “back mutated”. In certain such embodiments, the humanized antibody is mutated to include one or more of the amino acid residues found in the donor antibody. See, e.g., Saldanha et ai, MoI Immunol 36:709-19 (1999).

In certain embodiments the complementarity determining regions (CDRs) of the light and heavy chain variable regions of an antibody to TrkA can be grafted to framework regions (FRs) from the same, or another, species. In certain embodiments, the CDRs of the light and heavy chain variable regions of an antibody to TrkA can be grafted to consensus human FRs. To create consensus human FRs, in certain embodiments, FRs from several human heavy chain or light chain amino acid sequences are aligned to identify a consensus amino acid sequence. In certain embodiments, the FRs of an antibody to TrkA heavy chain or light chain are replaced with the FRs from a different heavy chain or light chain. In certain embodiments, rare amino acids in the FRs of the heavy and light chains of an antibody to TrkA are not replaced, while the rest of the FR amino acids are replaced. Rare amino acids are specific amino acids that are in positions in which they are not usually found in FRs. In certain embodiments, the grafted variable regions from an antibody to TrkA can be used with a constant region that is different from the constant region of an antibody to TrkA. In certain embodiments, the grafted variable regions are part of a single chain Fv antibody. CDR grafting is described, e.g., in U.S. Pat. Nos. 6,180,370, 6,054,297, 5,693,762, 5,859,205, 5,693,761, 5,565,332, 5,585,089, and 5,530,101, and in Jones et al, Nature, 321: 522-525 (1986); Riechmann et al, Nature, 332: 323-327 (1988); Verhoeyen et al, Science, 239:1534-1536 (1988), Winter, FEBS Letts., 430:92-94 (1998), which are hereby incorporated by reference for any purpose.

As described herein, an antibody or its antigen-binding fragment that binds to TrkA can comprise a human (i.e., fully human) antibody and/or part thereof. In certain embodiments, nucleotide sequences encoding, and amino acid sequences comprising, heavy and light chain immunoglobulin molecules, particularly sequences corresponding to the variable regions are provided. In certain embodiments, sequences corresponding to complementarity determining regions (CDR's), specifically from CDR1 through CDR3, are provided. According to certain embodiments, a hybridoma cell line expressing such an immunoglobulin molecule is provided. According to certain embodiments, a hybridoma cell line expressing such a monoclonal antibody is provided. In certain embodiments a hybridoma cell line is selected from at least one of the cell lines described in Table 1.

One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in WO 98/24893, U.S. Pat. No. 5,545,807, EP 546073, and EP 546073.

In certain embodiments, one can use constant regions from species other than human along with the human variable region(s).

The ability to clone and reconstruct megabase sized human loci in yeast artificial chromosomes (YACs) and to introduce them into the mouse germline provides an approach to elucidating the functional components of very large or crudely mapped loci as well as generating useful models of human disease. Furthermore, the utilization of such technology for substitution of mouse loci with their human equivalents could provide insights into the expression and regulation of human gene products during development, their communication with other systems, and their involvement in disease induction and progression.

Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

Humanized antibodies are those antibodies that, while initially starting off containing antibody amino acid sequences that are not human, have had at least some of these nonhuman antibody amino acid sequences replaced with human antibody sequences. This is in contrast with human antibodies, in which the antibody is encoded (or capable of being encoded) by genes possessed a human.

Other antibodies that are provided are variants of the antibody or the antigen-binding fragment thereof listed above formed by combination or subparts of the variable heavy and variable light chains shown in Table 1 and comprise variable light and/or variable heavy chains that each have at least 50%, 50%-60%, 60%-70%, 70%-80%, 80%-85%, 85%-90%, 90%-95%, 95%-97%, 97%-99%, or above 99% identity to the amino acid sequences of the sequences in Table 1 (either the entire sequence or a subpart of the sequence, e.g., one or more CDR). In some instances, such antibodies include at least one heavy chain and one light chain, whereas in other instances the variant forms contain two identical light chains and two identical heavy chains (or subparts thereof). In some embodiments, sequence comparison can be used in order to identify sections of the antibodies that can be modified by observing those variations that impact binding and those variations that do not appear to impact binding. For example, by comparing similar sequences, one can identify those sections (e.g., particular amino acids) that can be modified and how they can be modified while still retaining (or improving) the functionality of the antibody or the antigen-binding fragment thereof. In some embodiments, variants of the antibody include consensus groups and sequences between alternatives, as described above. The CDRs shown in Table 2 are defined based upon the IMGT method.

In certain embodiments, the antibody or its antigen-binding fragment comprises a heavy chain comprising a variable region comprising an amino acid sequence at least 90% identical to an amino acid sequence selected from at least one of the sequences of SEQ ID NO: 7, SEQ ID NO: 8, SEQ ID NO: 9, SEQ ID NO: 10, SEQ ID NO: 17, SEQ ID NO: 18, SEQ ID NO: 50 and SEQ ID NO: 52. In certain embodiments, the antibody or its antigen-binding fragment comprises a heavy chain comprising a variable region comprising an amino acid sequence at least 95% identical to an amino acid sequence selected from at least one of the sequences of SEQ ID NO: 7, SEQ ID NO: 8, SEQ ID NO: 9, SEQ ID NO: 10, SEQ ID NO: 17, SEQ ID NO: 18, SEQ ID NO: 50 and SEQ ID NO: 52. In certain embodiments, the antibody or its antigen-binding fragment comprises a heavy chain comprising a variable region comprising an amino acid sequence at least 99% identical to an amino acid sequence selected from at least one of the sequences of SEQ ID NO: 7, SEQ ID NO: 8, SEQ ID NO: 9, SEQ ID NO: 10, SEQ ID NO: 17, SEQ ID NO: 18, SEQ ID NO: 50 and SEQ ID NO: 52.

In some embodiments, the antibody or its antigen-binding fragment comprises a sequence that is at least 90%, 90-95%, and/or 95-99% identical to one or more CDRs from the CDRs in at least one of sequences of SEQ ID NO: 7, SEQ ID NO: 8, SEQ ID NO: 9, SEQ ID NO: 10, SEQ ID NO: 17, SEQ ID NO: 18, SEQ ID NO: 50 and SEQ ID NO: 52. In some embodiments, 1, 2, 3, 4, 5, or 6 CDR (each being at least 90%, 90-95%, and/or 95-99% identical to the above sequences) is present.

In some embodiments, the antibody or its antigen-binding fragment comprises a sequence that is at least 90%, 90-95%, and/or 95-99% identical to one or more FRs from the FRs in at least one of sequences of SEQ ID NO: 7, SEQ ID NO: 8, SEQ ID NO: 9, SEQ ID NO: 10, SEQ ID NO: 17, SEQ ID NO: 18, SEQ ID NO: 50 and SEQ ID NO: 52. In some embodiments, 1, 2, 3, or 4 FR (each being at least 90%, 90-95%, and/or 95-99% identical to the above sequences) is present.

In certain embodiments, the antibody or its antigen-binding fragment comprises a light chain comprising a variable region comprising an amino acid sequence at least 90% identical to an amino acid sequence selected from at least one of the sequences of SEQ ID NO: 14, SEQ ID NO: 20, SEQ ID NO: 51 and SEQ ID NO: 53. In certain embodiments, the antibody or its antigen-binding fragment comprises a light chain comprising a variable region comprising an amino acid sequence at least 95% identical to an amino acid sequence selected from at least one of the sequences of SEQ ID NO: 14, SEQ ID NO: 20, SEQ ID NO: 51 and SEQ ID NO: 53. In certain embodiments, an antigen-binding fragment thereof comprises a light chain comprising a variable region comprising an amino acid sequence at least 99% identical to an amino acid sequence selected from at least one of the sequences of SEQ ID NO: 14, SEQ ID NO: 20, SEQ ID NO: 51 and SEQ ID NO: 53.

In some embodiments, the antibody or its antigen-binding fragment comprises a sequence that is at least 90%, 90-95%, and/or 95-99% identical to one or more CDRs from the CDRs in at least one of sequences of SEQ ID NO: 14, SEQ ID NO: 20, SEQ ID NO: 51 and SEQ ID NO: 53. In some embodiments, 1, 2, 3, 4, 5, or 6 CDR (each being at least 90%, 90-95%, and/or 95-99% identical to the above sequences) is present.

In some embodiments, the antibody or its antigen-binding fragment comprises a sequence that is at least 90%, 90-95%, and/or 95-99% identical to one or more FRs from the FRs in at least one of sequences of SEQ ID NO: 14, SEQ ID NO: 20, SEQ ID NO: 51 and SEQ ID NO: 53. In some embodiments, 1, 2, 3, or 4 FR (each being at least 90%, 90-95%, and/or 95-99% identical to the above sequences) is present.

In light of the present disclosure, a skilled artisan will be able to determine suitable variants of the antibody or the antigen-binding fragment thereof as set forth herein using well-known techniques. In certain embodiments, one skilled in the art can identify suitable areas of the molecule that may be changed without destroying activity by targeting regions not believed to be important for activity. In certain embodiments, one can identify residues and portions of the molecules that are conserved among similar polypeptides. In certain embodiments, even areas that can be important for biological activity or for structure can be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a protein that correspond to amino acid residues which are important for activity or structure in similar proteins. One skilled in the art can opt for chemically similar amino acid substitutions for such predicted important amino acid residues.

One skilled in the art can also analyze the three-dimensional structure and amino acid sequence in relation to that structure in similar antibodies. In view of such information, one skilled in the art can predict the alignment of amino acid residues of an antibody with respect to its three-dimensional structure. In certain embodiments, one skilled in the art can choose not to make radical changes to amino acid residues predicted to be on the surface of the protein, since such residues can be involved in important interactions with other molecules. Moreover, one skilled in the art can generate test variants containing a single amino acid substitution at each desired amino acid residue. The variants can then be screened using activity assays known to those skilled in the art. Such variants can be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

In certain embodiments, antigen-binding fragment thereof variants include glycosylation variants wherein the number and/or type of glycosylation site has been altered compared to the amino acid sequences of a parent polypeptide. In certain embodiments, protein variants comprise a greater or a lesser number of N-linked glycosylation sites than the native protein. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants can be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

According to certain embodiments, amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physicochemical or functional properties on such polypeptides. According to certain embodiments, single or multiple amino acid substitutions (in certain embodiments, conservative amino acid substitutions) can be made in the naturally occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden & J. Tooze, eds., Garland Publishing, New York, N. Y. (1991)); and Thornton et al, Nature, 354:105 (1991), which are each incorporated herein by reference.

In some embodiments, the variants are variants of the nucleic acid sequences of the antibody or the antigen-binding fragment thereof disclosed herein. One of skill in the art will appreciate that the above discussion can be used for identifying, evaluating, and/creating antibodies and protein variants and also for nucleic acid sequences that can encode for those protein variants. Thus, nucleic acid sequences encoding for those protein variants (as well as nucleic acid sequences that encode for the antibody or the antigen-binding fragment thereof in Table 1, but are different from those explicitly disclosed herein) are contemplated.

In certain embodiments, the antibody, or the antigen-binding fragment thereof are produced by immunization with an antigen (e.g., TrkA). In certain embodiments, antibodies can be produced by immunization with full-length TrkA, a soluble form of TrkA, the extracellular domain alone, a splice variant form of TrkA, or a fragment thereof. In certain embodiments, the antibodies of the present disclosure can be polyclonal or monoclonal, and/or can be recombinant antibodies. In certain embodiments, antibodies of the present disclosure are human antibodies prepared, for example, by immunization of transgenic animals capable of producing human antibodies (see, for example, PCT Application No. WO 93/12227).

In certain embodiments, certain strategies can be employed to manipulate inherent properties of an antibody, such as the affinity of an antibody for its target. Such strategies include, but are not limited to, the use of site-specific or random mutagenesis of the polynucleotide molecule encoding an antibody to generate an antibody variant. In certain embodiments, such generation is followed by screening for antibody variants that exhibit the desired change, e.g. increased or decreased affinity.

In certain embodiments, the amino acid residues targeted in mutagenic strategies are those in the CDRs. In certain embodiments, amino acids in the framework regions of the variable domains are targeted. In certain embodiments, such framework regions have been shown to contribute to the target binding properties of certain antibodies. See, e.g., Hudson, Curr. Opin. Biotech., 9:395-402 (1999) and references therein.

As will be appreciated, antibodies can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Cell lines of particular preference are selected through determining which cell lines have high expression levels and produce antibodies with constitutive TrkA binding properties.

In certain embodiments, the antibody, or the antigen-binding fragment thereof comprises an immunoglobulin molecule of at least one of the IgG1, IgG2, IgG3, IgG4, Ig E, IgA, IgD, and IgM isotype. In certain embodiments, the antibody, or the antigen-binding fragment thereof comprises a human kappa light chain and/or a human heavy chain. In certain embodiments, the heavy chain is of the IgG1, IgG2, IgG3, IgG4, IgE, IgA, IgD, or IgM isotype. In certain embodiments, the antibody, or the antigen-binding fragment thereof has been cloned for expression in mammalian cells. In certain embodiments, the antibody, or the antigen-binding fragment thereof comprises a constant region other than any of the constant regions of the IgG1, IgG2, IgG3, IgG4, IgE, IgA, IgD, and IgM isotype.

Epitopes to which anti-TrkA antibodies bind are provided. In some embodiments, epitopes that are bound by the presently disclosed antibodies are particularly useful. In some embodiments, the antibody, or the antigen-binding fragment thereof that binds to any of the epitopes that are bound by the antibodies described herein are useful. In some embodiments, the epitopes bound by any of the antibodies listed in Table 1 are especially useful. In some embodiments, the epitope is on the extracellular domain of TrkA.

In some embodiments, the antibody or the antigen-binding fragment thereof may capable of competing with a reference antibody for binding to the TrkA, wherein the reference antibody comprises LCDR1-3 and HCDR1-3, said LCDR1 comprises the amino acid sequence as set forth in SEQ ID NO: 11, said LCDR2 comprises the amino acid sequence as set forth in SEQ ID NO: 12 (RT), said LCDR3 comprises the amino acid sequence as set forth in SEQ ID NO: 47, said HCDR1 comprises the amino acid sequence as set forth in SEQ ID NO: 43, said HCDR2 comprises the amino acid sequence as set forth in SEQ ID NO: 44, and said HCDR3 comprises the amino acid sequence as set forth in SEQ ID NO: 45.

In some embodiments, the reference antibody comprises VH and VL, wherein the VH comprises the amino acid sequence as set forth in SEQ ID NO: 54, the VL comprises the amino acid sequence as set forth in SEQ ID NO: 55.

In some embodiments, the antibody or the antigen-binding fragment thereof may comprise HCDR1-3 and LCDR1-3, wherein the HCDR1 comprises the amino acid sequence as set forth in SEQ ID NO: 43, the HCDR2 comprises the amino acid sequence as set forth in SEQ ID NO: 44, the HCDR3 comprises the amino acid sequence as set forth in SEQ ID NO: 45, the LCDR1 comprises the amino acid sequence as set forth in SEQ ID NO: 11, the LCDR2 comprises the amino acid sequence as set forth in SEQ ID NO: 12 (RT), and the LCDR3 comprises the amino acid sequence as set forth in SEQ ID NO: 47.

In some embodiments, the antibody or the antigen-binding fragment thereof may comprise HCDR1-3 and LCDR1-3, wherein the HCDR1 comprises an amino acid sequence as set forth in SEQ ID NO: 1, the HCDR2 comprises an amino acid sequence as set forth in SEQ ID NO: 2, the HCDR3 comprises an amino acid sequence as set forth in SEQ ID NO: 6, the LCDR1 comprises an amino acid sequence as set forth in SEQ ID NO: 11, the LCDR2 comprises an amino acid sequence as set forth in SEQ ID NO: 12 (RT), and the LCDR3 comprises an amino acid sequence as set forth in SEQ ID NO: 13.

In some embodiments, the antibody or the antigen-binding fragment thereof may comprise HCDR1-3 and LCDR1-3, wherein the HCDR1 comprises an amino acid sequence as set forth in SEQ ID NO: 1, the HCDR2 comprises an amino acid sequence as set forth in SEQ ID NO: 3, the HCDR3 comprises an amino acid sequence as set forth in SEQ ID NO: 6, the LCDR1 comprises an amino acid sequence as set forth in SEQ ID NO: 11, the LCDR2 comprises an amino acid sequence as set forth in SEQ ID NO: 12 (RT), and the LCDR3 comprises an amino acid sequence as set forth in SEQ ID NO: 13.

In some embodiments, the antibody or the antigen-binding fragment thereof may comprise HCDR1-3 and LCDR1-3, wherein the HCDR1 comprises an amino acid sequence as set forth in SEQ ID NO: 1, the HCDR2 comprises an amino acid sequence as set forth in SEQ ID NO: 4, the HCDR3 comprises an amino acid sequence as set forth in SEQ ID NO: 6, the LCDR1 comprises an amino acid sequence as set forth in SEQ ID NO: 11, the LCDR2 comprises an amino acid sequence as set forth in SEQ ID NO: 12 (RT), and the LCDR3 comprises an amino acid sequence as set forth in SEQ ID NO: 13.

In some embodiments, the antibody or the antigen-binding fragment thereof may comprise HCDR1-3 and LCDR1-3, wherein the HCDR1 comprises an amino acid sequence as set forth in SEQ ID NO: 1, the HCDR2 comprises an amino acid sequence as set forth in SEQ ID NO: 5, the HCDR3 comprises an amino acid sequence as set forth in SEQ ID NO: 6, the LCDR1 comprises an amino acid sequence as set forth in SEQ ID NO: 11, the LCDR2 comprises an amino acid sequence as set forth in SEQ ID NO: 12 (RT), and the LCDR3 comprises an amino acid sequence as set forth in SEQ ID NO: 13.

In some embodiments, the antibody or the antigen-binding fragment thereof may comprise HCDR1-3 and LCDR1-3, wherein the HCDR1 comprises an amino acid sequence as set forth in SEQ ID NO: 15, the HCDR2 comprises an amino acid sequence as set forth in SEQ ID NO: 3, the HCDR3 comprises an amino acid sequence as set forth in SEQ ID NO: 16, the LCDR1 comprises an amino acid sequence as set forth in SEQ ID NO: 11, the LCDR2 comprises an amino acid sequence as set forth in SEQ ID NO: 12 (RT), and the LCDR3 comprises an amino acid sequence as set forth in SEQ ID NO: 19.

In some embodiments, the antibody or the antigen-binding fragment thereof may comprise HCDR1-3 and LCDR1-3, wherein the HCDR1 comprises an amino acid sequence as set forth in SEQ ID NO: 15, the HCDR2 comprises an amino acid sequence as set forth in SEQ ID NO: 5, the HCDR3 comprises an amino acid sequence as set forth in SEQ ID NO: 16, the LCDR1 comprises an amino acid sequence as set forth in SEQ ID NO: 11, the LCDR2 comprises an amino acid sequence as set forth in SEQ ID NO: 12 (RT), and the LCDR3 comprises an amino acid sequence as set forth in SEQ ID NO: 19.

In some embodiments, the antibody or the antigen-binding fragment thereof may comprise HCDR1-3 and LCDR1-3, wherein the HCDR1 comprises an amino acid sequence as set forth in SEQ ID NO: 15, the HCDR2 comprises an amino acid sequence as set forth in SEQ ID NO: 5, the HCDR3 comprises an amino acid sequence as set forth in SEQ ID NO: 6, the LCDR1 comprises an amino acid sequence as set forth in SEQ ID NO: 11, the LCDR2 comprises an amino acid sequence as set forth in SEQ ID NO: 12 (RT), and the LCDR3 comprises an amino acid sequence as set forth in SEQ ID NO: 19.

In some embodiments, the antibody or the antigen-binding fragment thereof may comprise VH and VL, wherein the VH comprises the amino acid sequence as set forth in SEQ ID NO: 54, and the VL comprises the amino acid sequence as set forth in SEQ ID NO: 55.

In some embodiments, the antibody or the antigen-binding fragment thereof may comprise VH and VL, wherein the VH comprises the amino acid sequence as set forth in SEQ ID NO: 46, and the VL comprises the amino acid sequence as set forth in SEQ ID NO: 48.

In some embodiments, the antibody or the antigen-binding fragment thereof may comprise VH and VL, wherein the VH comprises an amino acid sequence as set forth in SEQ ID NO: 7, the VL comprises an amino acid sequence as set forth in SEQ ID NO: 14.

In some embodiments, the antibody or the antigen-binding fragment thereof may comprise VH and VL, wherein the VH comprises an amino acid sequence as set forth in SEQ ID NO: 8, the VL comprises an amino acid sequence as set forth in SEQ ID NO: 14.

In some embodiments, the antibody or the antigen-binding fragment thereof may comprise VH and VL, wherein the VH comprises an amino acid sequence as set forth in SEQ ID NO: 9, the VL comprises an amino acid sequence as set forth in SEQ ID NO: 14.

In some embodiments, the antibody or the antigen-binding fragment thereof may comprise VH and VL, wherein the VH comprises an amino acid sequence as set forth in SEQ ID NO: 10, the VL comprises an amino acid sequence as set forth in SEQ ID NO: 14.

In some embodiments, the antibody or the antigen-binding fragment thereof may comprise VH and VL, wherein the VH comprises an amino acid sequence as set forth in SEQ ID NO: 17, the VL comprises an amino acid sequence as set forth in SEQ ID NO: 20.

In some embodiments, the antibody or the antigen-binding fragment thereof may comprise VH and VL, wherein the VH comprises an amino acid sequence as set forth in SEQ ID NO: 18, the VL comprises an amino acid sequence as set forth in SEQ ID NO: 20.

In some embodiments, the antibody or the antigen-binding fragment thereof may comprise VH and VL, wherein the VH comprises an amino acid sequence as set forth in SEQ ID NO: 50, the VL comprises an amino acid sequence as set forth in SEQ ID NO: 51.

In some embodiments, the antibody or the antigen-binding fragment thereof may comprise VH and VL, wherein the VH comprises an amino acid sequence as set forth in SEQ ID NO: 52, the VL comprises an amino acid sequence as set forth in SEQ ID NO: 53.

The antibody or antigen-binding fragment may also encompass a homologue or a variant thereof having substantially the same function/property thereto. In some cases, the homologue or variant may have an amino acid sequence different from that of the antibody or antigen-binding fragment of the present disclosure by at least one amino acid. For example, the homologue or variant may be a polypeptide different from the antibody or the antigen-binding fragment thereof by an addition, deletion or substitution of one or more amino acid, such as 1-50, 1-40, 1-30, 1-20, 1-15, 1-14, 1-13, 1-12, 1-11, 1-10, 1-9, 1-8, 1-7, 1-6, 1-5, 1-4, 1-3, or 1-2 amino acids. In some cases, the homologue or variant may be a polypeptide having a sequence identity of at least 80% with the antibody or antigen-binding fragment thereof. For example, the homologue or variant may be a polypeptide having a sequence identity of 80% (e.g., at least 85%, at least 90%, at least 91%, at least 92%, at least 93%, at least 94%, at least 95%, at least 96%, at least 97%, at least 98%, at least 99% or higher) to the antibody or antigen-binding fragment thereof.

The term “percent (%) sequence identity,” as used in the context of polypeptide sequences identified herein, generally refers to the percentage of amino acid residues or nucleotides in a query sequence that are identical with the amino acid residues or nucleotides of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid/nucleotide sequence identity can be achieved in various ways that are within the skill in the art, for instance, using publicly available computer software such as BLAST, BLAST-2, ALIGN, NEEDLE or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide/polynucleotide sequence, or may be measured over a shorter length, for example, over the length of a fragment taken from a larger, defined polypeptide/polynucleotide sequence. It is understood that any fragment length supported by the sequences shown herein, in the tables, figures or Sequence Listing, may be used to describe a length over which percentage identity may be measured.

In the present application, the CDR sequences may be classified according to a specific CDR classification standard (for example, the IMGT method). It should be noted that if the amino acid sequence of a TrkA antibody or the antigen-binding fragment thereof determined according to a specific CDR classification standard (such as the IMGT method) is the same as the CDR sequence defined in the present application, then the TrkA antibody or the antigen-binding fragment thereof is also within the protection scope of the present application. According to different CDR classification standards, the precise identification of CDR positions may be slightly different, and therefore the present application comprises not only the CDRs shown in the sequence listing but also the CDRs comprised in the VH and VL domains using other classification methods for example, Chothia, enhanced Chothia, AbM, Kabat or IMGT.

In another aspect, the present application provides a fusion protein, comprising the antibody or the antigen-binding fragment thereof of the present disclosure.

In another aspect, the present application provides a protein conjugate (such as an immunoconjugate), comprising the antibody or the antigen-binding fragment thereof of the present disclosure.

### Nucleic Acid, Vector, Cell and Preparation Method

In another aspect, the present disclosure provides isolated nucleic acid or molecules, encoding for the antibody or the antigen-binding fragment thereof, or the fusion protein.

The isolated nucleic acids may comprise one or more nucleic acid molecules, with each encoding for at least a part of the antibody of the present disclosure or an antigen-binding fragment thereof. For example, the isolated nucleic acids may comprise at least two nucleic acid molecules, with one encoding for the antibody heavy chain or a fragment thereof, and one encoding for the antibody light chain or a fragment thereof. In some cases, the isolated nucleic acids may encode for a fusion protein.

The isolated nucleic acid or isolated nucleic acids may be synthesized using recombinant techniques well known in the art. For example, the isolated nucleic acid or isolated nucleic acids may be synthesized with an automated DNA synthesizer. Standard recombinant DNA and molecular cloning techniques include those described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987). Briefly, the subject nucleic acids may be prepared from genomic DNA fragments, cDNAs, and RNAs, all of which may be extracted directly from a cell or recombinantly produced by various amplification processes including but not limited to PCR and RT-PCR.

Direct chemical synthesis of nucleic acids typically involves sequential addition of 3′-blocked and 5′-blocked nucleotide monomers to the terminal 5′-hydroxyl group of a growing nucleotide polymer chain, wherein each addition is affected by nucleophilic attack of the terminal 5′-hydroxyl group of the growing chain on the 3′-position of the added monomer, which is typically a phosphorus derivative, such as a phosphotriester, phosphoramidite, or the like. See for example, Matteuci et al., Tet. Lett. 521:719 (1980); U.S. Pat. No. 4,500,707 to Caruthers et al.; and U.S. Pat. Nos. 5,436,327 and 5,700,637 to Southern et al.

In another aspect, the present disclosure provides a vector or vectors, comprising the isolated nucleic acid molecule or molecules.

The vector may be any linear nucleic acids, plasmids, phagemids, cosmids, RNA vectors, viral vectors and the like. Non-limiting examples of a viral vector may include a retrovirus, an adenovirus and an adeno-associated virus. In some cases, the vector is an expression vector, e.g. a phage display vector.

An expression vector may be suitable for use in particular types of host cells and not others. For example, the expression vector can be introduced into the host organism, which is then monitored for viability and expression of any genes/polynucleotides contained in the vector.

The expression vector may also contain one or more selectable marker genes that, upon expression, confer one or more phenotypic traits useful for selecting or otherwise identifying host cells that carry the expression vector. Non-limiting examples of suitable selectable markers for eukaryotic cells include dihydrofolate reductase and neomycin resistance.

The subject vectors can be introduced into a host cell stably or transiently by a variety of established techniques. For example, one method involves a calcium chloride treatment wherein the expression vector is introduced via a calcium precipitate. Other salts, for example calcium phosphate, may also be used following a similar procedure. In addition, electroporation (that is, the application of current to increase the permeability of cells to nucleic acids) may be used. Other examples of transformation methods include microinjection, DEAE dextran mediated transformation, and heat shock in the presence of lithium acetate. Lipid complexes, liposomes, and dendrimers may also be employed to transfect the host cells.

In another aspect, the present disclosure provides a cell (e.g., an isolated cell, such as a host cell), comprising the isolated nucleic acid molecule or molecules of the present disclosure or the vector or vectors of the present disclosure.

The cell may express the antibody, or the antigen-binding fragment thereof of the present disclosure, or the fusion protein of the present disclosure. The cell may be a eukaryotic cell or a prokaryotic cell. An appropriate cell may be transformed or transfected with the nucleic acid(s) or vector(s) of the present disclosure and utilized for the expression and/or secretion of the antibody, the antigen-binding fragment thereof, or the fusion protein. For example, the cell may be E. coli cells, other bacterial host cells, yeast cells, or various higher eukaryotic cells.
n another aspect, the present disclosure provides a method for producing the antibody or the antigen-binding fragment thereof, or the fusion protein of the present disclosure, comprising culturing the cell of the present disclosure under conditions enabling expression of the antibody, the antigen-binding fragment thereof, or the fusion protein.

The method optionally may further comprise harvesting the antibody or the antigen-binding fragment thereof, or the fusion protein of the present disclosure.

### Compositions

In another aspect, the present disclosure provides a composition, comprising the antibody or the antigen-binding fragment thereof, the fusion protein, the protein conjugate, the isolated nucleic acid molecule or molecules, the vector or vectors, and/or the cell of the present disclosure, and optionally a pharmaceutically acceptable excipient.

In some cases, the pharmaceutically acceptable excipient may comprise a buffer. In some cases, the pharmaceutically acceptable excipient may comprise an amino acid.

In some embodiments, the pH of the composition may be 1-13, for example, the pH may be about 1, 2, 3, 4, 5, 6, 7, 8, 9, 10, 11, 12, or 13.

In some cases, the composition may further comprise an effective amount of an additional therapeutically active component, for example, an additional therapeutically active component for treating a disease or a disorder. Each of the active components may be present in the pharmaceutical composition in a pharmaceutically active amount. In the composition, the antibody, the fragment thereof of the present application may or may not be associated with the additional active component.

Described below are non-limiting exemplary pharmaceutical compositions and methods for preparing the same. The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. In some cases, the pharmaceutical composition may be a liquid pharmaceutical composition.

Pharmaceutical compositions of the disclosure can be presented as discrete dosage forms, with each dosage containing a predetermined amount of an active ingredient as a powder or in granules, a solution, or a suspension in an aqueous or non-aqueous liquid. Such dosage forms can be prepared by any of the methods known to a skilled person, for example, it may include the step of bringing the active ingredient into association with the carrier, which constitutes one or more other ingredients. In general, the compositions are prepared by uniformly and intimately mixing the active ingredient with liquid carriers or finely divided solid carriers or both, and then, if necessary, shaping the product into the desired presentation.

The antibody, the antigen-binding fragment thereof, or the fusion protein of the present disclosure can be combined in an intimate admixture with a pharmaceutical carrier according to conventional pharmaceutical compounding techniques. The carrier can take a wide variety of forms depending on the form of preparation desired for administration.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and/or mixtures thereof.

The pharmaceutical compositions of the present disclosure may comprise a therapeutically effective amount of the active agent (e.g., the antibody, the antigen-binding fragment thereof, or the fusion protein of the present disclosure). A therapeutically effective amount is an amount of the subject pharmaceutical composition capable of preventing and/or curing (at least partially) a condition or disorder (e.g., a chronic pain) and/or any complications thereof in a subject suffering from or having a risk of developing said condition or disorder. The specific amount/concentration of the active agent comprised may vary according to the method of administration and the need of a patient, and can be determined based on e.g., volume, viscosity, and/or body weight of a patient etc. For example, an appropriate dosage may be about 0.1 mg or 1 mg/kg/day to about 50 mg/kg/day; sometimes, the dosage can be even higher. It shall be understood that these specific doses may be conveniently adjusted by a skilled person in the art (e.g., a doctor or a pharmacist) based on conditions of a specific patient, formulation, and/or disease.

### Medical Use and Methods of Treatment

In another aspect, the present disclosure provides a use of the antibody or the antigen-binding fragment thereof, the fusion protein, the protein conjugate, the isolated nucleic acid molecule or molecules, the vector or vectors, and/or the cell of the present disclosure in the manufacture of a medicament for preventing and/or treating a disease or disorder.

In a further aspect, the present application provides a method for preventing and/or treating a disease or disorder in a subject in need thereof, comprising administering to the subject an effective amount of the antibody or the antigen-binding fragment the fusion protein, the protein conjugate, the isolated nucleic acid molecule or molecules, the vector or vectors, the cell, and/or the composition of the present disclosure.

In a further aspect, the present application provides the antibody or the antigen-binding fragment the fusion protein, the protein conjugate, the isolated nucleic acid molecule or molecules, the vector or vectors, the cell, and/or the composition of the present disclosure, for use in preventing and/or treating a disease or disorder.

For example, the disease or disorder may be tumors, peripheral neuropathies, ophthalmic diseases, diabetes and related complications, cardiovascular disease, and/or Alzheimer's disease.

For example, the tumors may be neuronal tumors or non-neuronal tumors.

For example, the tumors may be neuroblastoma, glioblastoma, medulloblastoma, prostatic cancer, epithelial ovarian cancer, non-small cell lung cancer, and/or breast cancer.

For example, the peripheral neuropathies may be chemotherapy-induced peripheral neuropathy (CIPN), and/or diabetic neuropathy.

For example, the ophthalmic diseases may be dry eye disease (DED).

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 TrkA-ECD Recombinant Protein Preparation

To construct the expression plasmid, the gene of human TrkA extracellular domain consisting of the amino acid (AA) 33-423 (SEQ ID NO: 42) was synthesized and subcloned into pcDNA3.4 vector with C-terminus fused to a His tag and N-terminus fused to a signal peptide and a flag tag. The plasmid was transiently transfected into 293-F cells, and cultured for 5-7 days in a CO2 incubator equipped with rotating shaker. The supernatant was harvested and purified through one-step immobilized metal affinity chromatography. The purified protein was then buffer-exchanged into phosphate buffered saline (PBS).

### Example 2 Hybridoma Antibody Generation

BALB/c mice (5 mice in each group) were immunized with full-length TrkA ECD protein, as shown in Table 1. In brief. TrkA ECD protein was mixed with Complete Freund's adjuvant (CFA) or Incomplete Freund's adjuvant (IFA) in a 1:1 ratio to prepare a stable oil-in-water emulsion. After immunization, serum samples were taken to determine the antibody response (titer) by indirect ELISA and FACS. Four days after the final immunization, the splenocytes were isolated and fused with SP2/0 myeloma cells by electro-cell fusion to induce cell fusion and form hybridomas.

TABLE 3
Immunization Schedule

| Procedure | Schedule | Route | Dosage |
|---|---|---|---|
| Pre-Immune Bleed | T = −4 days | | |
| Primary | T = 0 days | s.c. injection | 50 ug TrkA |
| Immunization | | | ECD/mouse |
| 1^{st}Boost | T = 14 days | i.p. injection | 25 ug TrkA |
| | | | ECD/mouse |
| Test Bleed 1 | T = 21 days | | |
| 2^{nd}Boost | T = 28 days | s.c. injection | 25 ug TrkA |
| | | | ECD/mouse |
| Test Bleed 2 | T = 35 days | | |
| Final Boost | T = 56 ± 7 days | i.p. injection | 25 ug TrkA |
| | | | ECD/mouse |
| Cell Fusion | T = Final | | |
| | boost + 4 days | | |

The binding of hybridoma supernatants or purified antibodies with TrkA ECD was determined by ELISA. The ELISA-positive antibody clones were further verified by fluorescence-activated cell sorting (FACS) assay. The human TrkA overexpressed CellSensor® TrkA-NFAT-bla CHO-K1 cell (Thermo Fisher, K1516) was used in FACS.

Hybridoma fusion clones which both showed positive on ELISA and FACS assays and did not react with TrkB, TrkC and P75 were selected for further screening by TrkA-activation dependent NFAT-promoter driven reporter assay following Invitrogen's CellSensor TrkA-NFAT-bla CHO-K1 cell-based assay protocol. Briefly, harvest cells and resuspend in Assay Medium to a density of 375, 000 cells/mL, then add Assay Medium or the cell suspension to each testing well. Incubate the plates in a 37 °C/5% CO₂ incubator for 16 hours. Add 8 μl of NGF or Diluted antibody samples to testing wells. Incubate the plate in a humidified 37 °C/5% CO₂ incubator for 5 hours. Prepare 6× LiveBLAzer™-FRET B/G (CCF4-AM) Substrate Mixture before use. Remove the assay plate from the incubator and allow the plate to equilibrate to room temperature. Add 8 μL of 6× Substrate Mixture to each well. Incubate at room temperature for 2 hours. At last, set the fluorescence plate reader to bottom-read mode, read the wells using a dual emission wavelength measurement protocol (measurement 1: 405 nm excitation & 460 nm emission; measurement 2: 405 nm excitation & 530 nm emission).

The ELISA and FACS binding positive clones were also tested by TrkA-activation dependent Akt phosphorylation by HTRF assay following Cisbio's pAKT(S473) assay manual. Briefly, Harvest cells and resuspend in Assay Medium to a density of 4×10⁵ cells/ml. Then plate 100 μl of cells in 96-well culture-treated plates. Incubate the plates in a 37 °C/5% CO₂ incubator for 16 hours. Add 100 μl of NGF or Diluted antibody samples to testing wells. Incubate the assay plate in a humidified 37 °C/5% CO2 incubator for 30 minutes. Remove cell supernatant carefully, then immediately add 50 μl of supplemented lysis buffer (1×) and incubate for 30 minutes at room temperature under shaking. After homogenization by pipetting up and down, transfer 16 μl cell lysate from the 96-well plate to a 384-well plate. Add 4 ul of premixed antibody to all wells. Cover the plate with a plate sealer and incubate for at least 4 hours at room temperature before reading. Set up the plate reader for Eu3+ Cryptate and read the fluorescence emission at two different wavelengths (665 nm and 620 nm) on a compatible HTRF® reader.

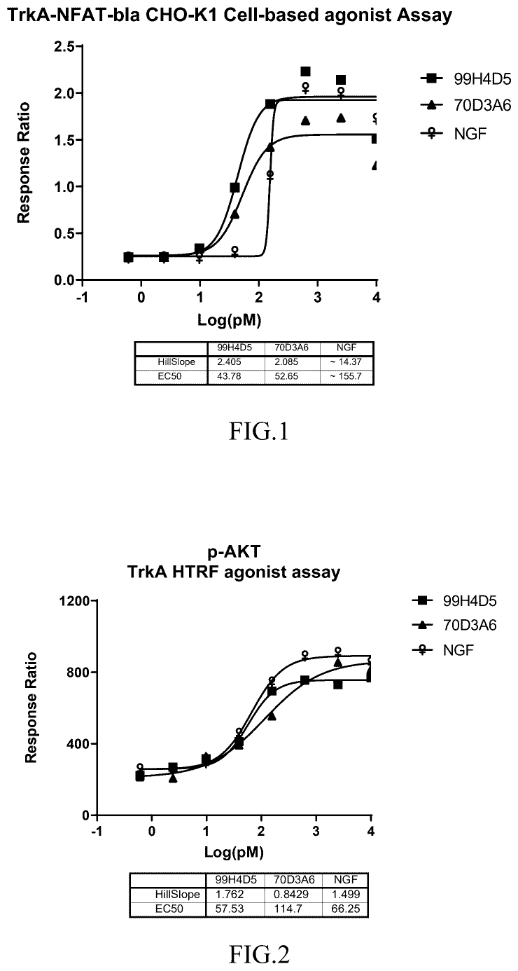

This procedure led to the identification of multiple hybridoma clones expressing human TrkA-specific antibodies and with agonistic activity (FIG. 1-2). The amino acid sequence of VH of Chi99H4D5 is as set forth in SEQ ID NO: 7, and the amino acid sequence of VL of Chi99H4D5 is as set forth in SEQ ID NO: 14. The amino acid sequence of VH of Chi70D3A6 is as set forth in SEQ ID NO: 17, and the amino acid sequence of VL of Chi70D3A6 is as set forth in SEQ ID NO: 20.

### Example 3 Cloning of Antibody Variable Region Gene

Hybridoma cells were cultured in a 10 cm dish and were collected at the logarithmic growth phase. Total cell RNA was extracted with Trizol (Invitrogen, 15596-018) following the manufacturer's introduction. The RNA was resuspended in nuclease-free water. The RNA concentration was determined by using absorbance at 260 nm. To obtain the cDNA templates, 4 ug of each RNA was reverse transcribed using HiFiScript cDNA Synthesis Kit (CWBIO, CW2569). Then two rounds of PCR reactions were performed to clone the variable gene of hybridoma mAbs. First-round PCR was performed by using degenerate primers based on the sequences reported previously (Wang, Z et al, J Immunol Methods. 2000; 233(1-2):167-177). The PCR products were sequenced. The sequencing results were analyzed using IIMGT/V-QUEST web program (http://www.imgt.org/IMGT_vquest/analysis). To amply the new VH or VL fragment with a signal peptide, the 5′ upper primer starts from the signal peptide of the corresponding germline gene was used for the second round of PCR. The PCR product was directly sequenced. Antibody complement-determinant regions (CDRs) were identified based on Kabat, AbM and IIMGT system by using ANARCI tools (Dunbar J et. al Bioinformatics. 2016; 32(2):298-300).

The heavy and light chain variable region sequences of murine mAb are as follows:

Chi99H4D5 VH
(SEQ ID NO: 7)
EVKLVESGGGLVKPGGSLKLSCAASGFTFSYYAISWLRQTPEKRLEWVAS
ISGNGDTYYLDSVKGRFTISRDNARNILYLQMSSLRSEDTAMYYCARGVG
GNYFDYWGQGTTLTVSS

The CDRs in the VH of Chi99H4D5 are as shown below:

| CDR | | | |
|---|---|---|---|
| Classi- | | | |
| fication | CDR | Sequence | SEQ ID NO |
| AbM | HCDR1 | GFTFSYYAIS | SEQ ID NO: |
| | | | 21 |
| | HCDR2 | SISGNGDTY | SEQ ID NO: |
| | | | 22 |
| | HCDR3 | GVGGNYFDY | SEQ ID NO: |
| | | | 26 |
| Kabat | HCDR1 | YYAIS | SEQ ID NO: |
| | | | 33 |
| | HCDR2 | SISGNGDTYYLDSVKG | SEQ ID NO: |
| | | | 34 |
| | HCDR3 | GVGGNYFDY | SEQ ID NO: |
| | | | 26 |
| IMGT | HCDR1 | GFTFSYYA | SEQ ID NO: |
| | | | 1 |
| | HCDR2 | ISGNGDT | SEQ ID NO: |
| | | | 2 |
| | HCDR3 | ARGVGGNYFDY | SEQ ID NO: |
| | | | 6 |

The Variant 1 (V1) of CDRs in the VH of Chi99H4D5 are as shown below:

| CDR | | | |
|---|---|---|---|
| Classi- | | | |
| fication | CDR | Sequence | SEQ ID NO |
| AbM | HCDR1 | GFTFSYYAIS | SEQ ID NO: |
| | | | 21 |
| | HCDR2 | SISGGGDTY | SEQ ID NO: |
| | | | 23 |
| | HCDR3 | GVGGNYFDY | SEQ ID NO: |
| | | | 26 |
| Kabat | HCDR1 | YYAIS | SEQ ID NO: |
| | | | 33 |
| | HCDR2 | SISGGGDTYYLDSVKG | SEQ ID NO: |
| | | | 35 |
| | HCDR3 | GVGGNYEDY | SEQ ID NO: |
| | | | 26 |
| IMGT | HCDR1 | GFTFSYYA | SEQ ID NO: |
| | | | 1 |
| | HCDR2 | ISGGGDT | SEQ ID NO: |
| | | | 3 |
| | HCDR3 | ARGVGGNYFDY | SEQ ID NO: |
| | | | 6 |

The Variant 2 (V2) of CDRs in the VH of Chi99H4D5 are as shown below:

| CDR | | | |
|---|---|---|---|
| Classi- | | | |
| fication | CDR | Sequence | SEQ ID NO |
| AbM | HCDR1 | GFTFSYYAIS | SEQ ID NO: |
| | | | 21 |
| | HCDR2 | SISGNGDVY | SEQ ID NO: |
| | | | 24 |
| | HCDR3 | GVGGNYFDY | SEQ ID NO: |
| | | | 26 |
| Kabat | HCDR1 | YYAIS | SEQ ID NO: |
| | | | 33 |
| | HCDR2 | SISGNGDVYYLDSVKG | SEQ ID NO: |
| | | | 36 |
| | HCDR3 | GVGGNYFDY | SEQ ID NO: |
| | | | 26 |
| IMGT | HCDR1 | GFTFSYYA | SEQ ID NO: |
| | | | 1 |
| | HCDR2 | ISGNGDV | SEQ ID NO: |
| | | | 4 |
| | HCDR3 | ARGVGGNYEDY | SEQ ID NO: |
| | | | 6 |

The Variant 3 (V3) of CDRs in the VH of Chi99H4D5 are as shown below:

| CDR | | | |
|---|---|---|---|
| Classi- | | | |
| fication | CDR | Sequence | SEQ ID NO |
| AbM | HCDR1 | GFTFSYYAIS | SEQ ID NO: |
| | | | 21 |
| | HCDR2 | SISGGGDVY | SEQ ID NO: |
| | | | 25 |
| | HCDR3 | GVGGNYFDY | SEQ ID NO: |
| | | | 26 |
| Kabat | HCDR1 | YYAIS | SEQ ID NO: |
| | | | 33 |
| | HCDR2 | SISGGGDVYYLDSVKG | SEQ ID NO: |
| | | | 37 |
| | HCDR3 | GVGGNYFDY | SEQ ID NO: |
| | | | 26 |
| IMGT | HCDR1 | GFTFSYYA | SEQ ID NO: |
| | | | 1 |
| | HCDR2 | ISGGGDV | SEQ ID NO: |
| | | | 5 |
| | HCDR3 | ARGVGGNYFDY | SEQ ID NO: |
| | | | 6 |

Chi99H4D5 VL (SEQ ID NO: 14)
DIVMTQSPAIMSASPGEKVTISCSASSSVSYMYWYQQKPGSSPKPLIYRT
SNLASGVPARFSGSGSGTSYSLTISTMEAEDAATYYCQQYHSYTPITFGA
GTKLDLK

The CDRs in the VL of Chi99H4D5

| CDR | | | |
|---|---|---|---|
| Classi- | | | |
| fication | CDR | Sequence | SEQ ID NO |
| AbM | LCDR1 | SASSSVSYMY | SEQ ID NO: |
| | | | 27 |
| | LCDR2 | RTSNLAS | SEQ ID NO: |
| | | | 28 |
| | LCDR3 | QQYHSYTPIT | SEQ ID NO: |
| | | | 13 |
| Kabat | LCDR1 | SASSSVSYMY | SEQ ID NO: |
| | | | 27 |
| | LCDR2 | RTSNLAS | SEQ ID NO: |
| | | | 28 |
| | LCDR3 | QQYHSYTPIT | SEQ ID NO: |
| | | | 13 |
| IMGT | LCDR1 | SSVSY | SEQ ID NO: |
| | | | 11 |
| | LCDR2 | RT | SEQ ID NO: |
| | | | 12 |
| | LCDR3 | QQYHSYTPIT | SEQ ID NO: |
| | | | 13 |

Chi70D3A6 VH (SEQ ID NO: 17)
EVKLVESGGDLVKPGGSLKLSCTASGFTFTYYAMSWVRQTPEKRLEWVAT
ISGGGDTYYLDSVKGRFTISRDNAGNILYLQMSSLRSEDTAMYYCARGVG
GNFFDYWGQGSTLTVSS

The CDRs in the VH of Chi70D3A6 are as shown below:

| CDR | | | |
|---|---|---|---|
| Classi- | | | |
| fication | CDR | Sequence | SEQ ID NO |
| AbM | HCDR1 | GFTFTYYAMS | SEQ ID NO: |
| | | | 29 |
| | HCDR2 | TISGGGDTY | SEQ ID NO: |
| | | | 30 |
| | HCDR3 | GVGGNFFDY | SEQ ID NO: |
| | | | 32 |
| Kabat | HCDR1 | YYAMS | SEQ ID NO: |
| | | | 38 |
| | HCDR2 | TISGGGDTYYLDSVKG | SEQ ID NO: |
| | | | 39 |
| | HCDR3 | GVGGNFFDY | SEQ ID NO: |
| | | | 32 |
| IMGT | HCDR1 | GFTFTYYA | SEQ ID NO: |
| | | | 15 |
| | HCDR2 | ISGGGDT | SEQ ID NO: |
| | | | 3 |
| | HCDR3 | ARGVGGNFFDY | SEQ ID NO: |
| | | | 16 |

The Variant 1 (V1) of CDRs in the VH of Chi70D3A6 are as shown below:

| CDR | | | |
|---|---|---|---|
| Classi- | | | |
| fication | CDR | Sequence | SEQ ID NO |
| AbM | HCDR1 | GFTFTYYAMS | SEQ ID NO: |
| | | | 29 |
| | HCDR2 | TISGGGDVY | SEQ ID NO: |
| | | | 31 |
| | HCDR3 | GVGGNFFDY | SEQ ID NO: |
| | | | 32 |
| Kabat | HCDR1 | YYAMS | SEQ ID NO: |
| | | | 38 |
| | HCDR2 | TISGGGDVYYLDSVKG | SEQ ID NO: |
| | | | 40 |
| | HCDR3 | GVGGNFFDY | SEQ ID NO: |
| | | | 32 |
| IMGT | HCDR1 | GFTFTYYA | SEQ ID NO: |
| | | | 15 |
| | HCDR2 | ISGGGDV | SEQ ID NO: |
| | | | 5 |
| | HCDR3 | ARGVGGNFFDY | SEQ ID NO: |
| | | | 16 |

Chi70D3A6 VL (SEQ ID NO: 20)
QIVLTQSPAIMSASPGEKVTISCSASSSVSYMYWYQQKPGSSPKPFIYRT
SNLASGVPSRFSGSGSGTSYSLTVSTMEAEDAATYYCQLYHSYTPITFGA
GTKLELK

The CDRs in the VL of Chi70D3A6 are as shown below:

| CDR | | | |
|---|---|---|---|
| Classi- | | | |
| fication | CDR | Sequence | SEQ ID NO |
| AbM | LCDR1 | SASSSVSYMY | SEQ ID NO: |
| | | | 27 |
| | LCDR2 | RTSNLAS | SEQ ID NO: |
| | | | 28 |
| | LCDR3 | QL YHSYTPIT | SEQ ID NO: |
| | | | 19 |
| Kabat | LCDR1 | SASSSVSYMY | SEQ ID NO: |
| | | | 27 |
| | LCDR2 | RTSNLAS | SEQ ID NO: |
| | | | 28 |
| | LCDR3 | QLYHSYTPIT | SEQ ID NO: |
| | | | 19 |
| IMGT | LCDR1 | SSVSY | SEQ ID NO: |
| | | | 11 |
| | LCDR2 | RT | SEQ ID NO: |
| | | | 12 |
| | LCDR3 | QLYHSYTPIT | SEQ ID NO: |
| | | | 19 |

### Example 4 Expression and Purification of Monoclonal Antibodies

To produce antibodies from hybridoma cells, the positive hybridomas were adapted to the Hybridoma-SFM medium firstly, then the cells were cultured in the Hybridoma-SFM medium at 5% CO₂ and 37 °C in an incubator for 4 days. Cell viability yield and protein production can be assessed after cultivation.

To transient transfect HEK293 for antibody recombinant expression, the VH and VL gene were subcloned into the pCDNA3.4 vector with the constant region respectively, further confirmed by DNA sequencing. The 5 μg plasmids (3 g light chain and 2 g heavy chain) were diluted by 1 ml of Opti-MEM® I Reduced Serum Medium (GIBCO, 31985-070) and transfected into human 293F cells with 20 μg PEI. Cells weres cultured in serum-free medium (Shanghai opmbiosciences, OPM-293CD03) with 5% CO₂, 37 °C, 125 rpm/min. The feed medium was replenished on day 1 and then cultured for another 4 days.

The antibody was purified by using GenScript Protein A MagBeads according to the manufacturer's introduction. Briefly, the clarified supernatant was incubated with the MagBeads at room temperature rocking for 2 h. Then, the MagBead was washed three times with PBS buffer. The purified antibody was eluted from the beads with Elution Buffer (0.1 M glycine, pH 3.0) followed by neutralization with Neutralization Buffer (1 M Tris, pH 8.5).

### Example 5 Determining the Affinity of TrkA Antibodies

For determining the affinity of TrkA antibodies, bio-layer interferometry (BLI) method was used with the Octet Red384 instrument. Briefly, Fc-coated biosensor AHQ tips (ForteBio) were pre-wet in PBS with 0.1% w/v bovine serum albumin and 0.05% Tween-20 (assay buffer) for a minimum of 10 min in the pre-wetting plate. The purified antibodies at 100 nM in assay buffer were captured on AHQ biosensors (ForteBio) to obtain capture levels of −1 nm. Serial 2-fold dilutions (3.125 nM to 200 nM) of human TrkA ECD solution were used to determine association rates, and dissociation rates respectively. All measurements were corrected for baseline drift by subtracting a control sensor exposed to assay buffer only. Association rate constants (kon), dissociation rate constants (koff), and KD for each antibody were calculated by applying a 1:1 interaction model (fitting local, full) using the ForteBio data analysis software.

The binding affinities of the selected murine antibodies were listed in Table 4.

TABLE 4
The binding affinities of the selected murine antibodies

| | | | | VH | VL |
|---|---|---|---|---|---|
| | KD | Kon | Koff | SEQ | SEQ |
| | (M) | (1/Ms) | (1/s) | ID NO: | ID NO: |
| Chi70D3A6 | 7.71E−10 | 1.29E+06 | 9.98E−04 | 17 | 20 |
| Chi99H4D5 | 1.30E−09 | 8.52E+05 | 1.11E−03 | 7 | 14 |

### Example 6 TrkA Antibodies Humanization

Humanization of murine antibodies was carried out using CDR grafting, as previously reported. Briefly, the parental (murine antibody) variable region (VH and VL) frameworks were replaced with those of the selected human germline V and J genes of VH and VL. The germline genes were selected on the basis of the homology between the parental antibody and the germline V and J genes. Human HC germline genes IGHV3-23*04 and IGHJ4*01 were selected as FR donors for the humanization of Chi99H4D5 and Chi70D3A6 VH; Human LC germline genes IGKV1-39*01 and IGKJ2*01 were selected as FR donors for the humanization of Chi99H4D5 and Chi70D3A6 VL. Humanized antibody Hu70D3A6-V12 with T57V mutation in H-CDR2 and back mutation L46P in the light chain was selected as the lead humanized antibody. Humanized antibody Hu99H4D5-V6 with N54G and T57V mutation in H-CDR2 and back mutation L46P in the light chain was selected as the lead humanized antibody.

The CDRs, VH and VL sequences of the Hu70D3A6-V12 are as below.

VH of Hu70D3A6-V12: SEQ ID NO: 50
EVQLVESGGGLVKPGGSLRLSCAASGFTFTYYAMSWVRQAPGKGLEWVSSI
SGGGDVYYADSVKGRFTISRDNAKNTLYLQMNSLRAEDTAVYYCARGVGGN
YFDYWGQGTLVTVS

| CDR | | | |
|---|---|---|---|
| Classi- | | | |
| fication | CDR | Sequence | SEQ ID NO |
| AbM | HCDR1 | GFTFTYYAMS | SEQ ID NO: |
| | | | 29 |
| | HCDR2 | SISGGGDVY | SEQ ID NO: |
| | | | 25 |
| | HCDR3 | GVGGNYFDY | SEQ ID NO: |
| | | | 26 |
| Kabat | HCDR1 | YYAMS | SEQ ID NO: |
| | | | 38 |
| | HCDR2 | SISGGGDVYYADSVKG | SEQ ID NO: |
| | | | 49 |
| | HCDR3 | GVGGNYFDY | SEQ ID NO: |
| | | | 26 |
| IMGT | HCDR1 | GFTFTYYA | SEQ ID NO: |
| | | | 15 |
| | HCDR2 | ISGGGDV | SEQ ID NO: |
| | | | 5 |
| | HCDR3 | ARGVGGNYFDY | SEQ ID NO: |
| | | | 6 |

VL of Hu70D3A6-V12: SEQ ID NO: 51
DIQMTQSPSSLSASVGDRVTITCSASSSVSYMYWYQQKPGKAPKPLIYRT
SNLASGVPSRFSGSGSGTDFTLTISSLQPEDFATYYCQLYHSYTPITFGQ
GTKLEIK

| CDR | | | |
|---|---|---|---|
| Classi- | | | |
| fication | CDR | Sequence | SEQ ID NO |
| AbM | LCDR1 | SASSSVSYMY | SEQ ID NO: |
| | | | 27 |
| | LCDR2 | RTSNLAS | SEQ ID NO: |
| | | | 28 |
| | LCDR3 | QLYHSYTPIT | SEQ ID NO: |
| | | | 19 |
| Kabat | LCDR1 | SASSSVSYMY | SEQ ID NO: |
| | | | 27 |
| | LCDR2 | RTSNLAS | SEQ ID NO: |
| | | | 28 |
| | LCDR3 | QLYHSYTPIT | SEQ ID NO: |
| | | | 19 |
| IMGT | LCDR1 | SSVSY | SEQ ID NO: |
| | | | 11 |
| | LCDR2 | RT | SEQ ID NO: |
| | | | 12 |
| | LCDR3 | QLYHSYTPIT | SEQ ID NO: |
| | | | 19 |

The CDRs, VH and VL sequences of the humanized are Hu99H4D5-V6 as below:

VH of Hu99H4D5-V6: SEQ ID NO: 52
EVQLVESGGGLVKPGGSLRLSCAASGFTFSYYAISWVRQAPGKGLEWVSS
ISGGGDVYYADSVKGRFTISRDNAKNTLYLQMNSLRAEDTAVYYCARGVG
GNYFDYWGQGTLVTVSS

| CDR | | | |
|---|---|---|---|
| Classi- | | | |
| fication | CDR | Sequence | SEQ ID NO |
| AbM | HCDR1 | GFTFSYYAIS | SEQ ID NO: |
| | | | 21 |
| | HCDR2 | SISGGGDVY | SEQ ID NO: |
| | | | 25 |
| | HCDR3 | GVGGNYFDY | SEQ ID NO: |
| | | | 26 |
| Kabat | HCDR1 | YYAIS | SEQ ID NO: |
| | | | 33 |
| | HCDR2 | SISGGGDVYYADSVKG | SEQ ID NO: |
| | | | 49 |
| | HCDR3 | GVGGNYFDY | SEQ ID NO: |
| | | | 26 |
| IMGT | HCDR1 | GFTFSYYA | SEQ ID NO: |
| | | | 1 |
| | HCDR2 | ISGGGDV | SEQ ID NO: |
| | | | 5 |
| | HCDR3 | ARGVGGNYFDY | SEQ ID NO: |
| | | | 6 |

VL of Hu99H4D5-V6: SEQ ID NO: 53
DIQMTQSPSSLSASVGDRVTITCSASSSVSYMYWYQQKPGKAPKPLIYRT
SNLASGVPSRFSGSGSGTDFTLTISSLQPEDFATYYCQQYHSYTPITFGQ
GTKLEIK

| CDR | | | |
|---|---|---|---|
| Classi- | | | |
| fication | CDR | Sequence | SEQ ID NO |
| AbM | LCDR1 | SASSSVSYMY | SEQ ID NO: |
| | | | 27 |
| | LCDR2 | RTSNLAS | SEQ ID NO: |
| | | | 28 |
| | LCDR3 | QQYHSYTPIT | SEQ ID NO: |
| | | | 13 |
| Kabat | LCDR1 | SASSSVSYMY | SEQ ID NO: |
| | | | 27 |
| | LCDR2 | RTSNLAS | SEQ ID NO: |
| | | | 28 |
| | LCDR3 | QQYHSYTPIT | SEQ ID NO: |
| | | | 13 |
| IMGT | LCDR1 | SSVSY | SEQ ID NO: |
| | | | 11 |
| | LCDR2 | RT | SEQ ID NO: |
| | | | 12 |
| | LCDR3 | QQYHSYTPIT | SEQ ID NO: |
| | | | 13 |

The corresponding encoding nucleic acid sequences of Hu99H4D5-V6 and Hu70D3A6-V12 were synthesized and subcloned into a pCDNA3.4-hIgG4 or pCDNA3.4-hKappa vector. All recombinant antibodies were expressed and purified as described in Example 4. The affinity of antibodies were determined as described in Example 5. The results are shown in the table below, Hu99H4D5-V6 and Hu70D3A6-V12 had similar affinity as the chimeric antibody.

TABLE 5
The binding affinities of the humanized antibodies

| | | | | VH | VL |
|---|---|---|---|---|---|
| | | | | SEQ ID | SEQ ID |
| Sample ID | K_{D} (M) | Kₒₙ(1/Ms) | K_{off}(1/s) | NO. | NO. |
| Hu70D3A6-V12 | 5.91E−10 | 4.84E+05 | 2.86E−04 | 50 | 51 |
| Hu99H4D5-V6 | 7.53E−10 | 4.63E+05 | 3.49E−03 | 52 | 53 |

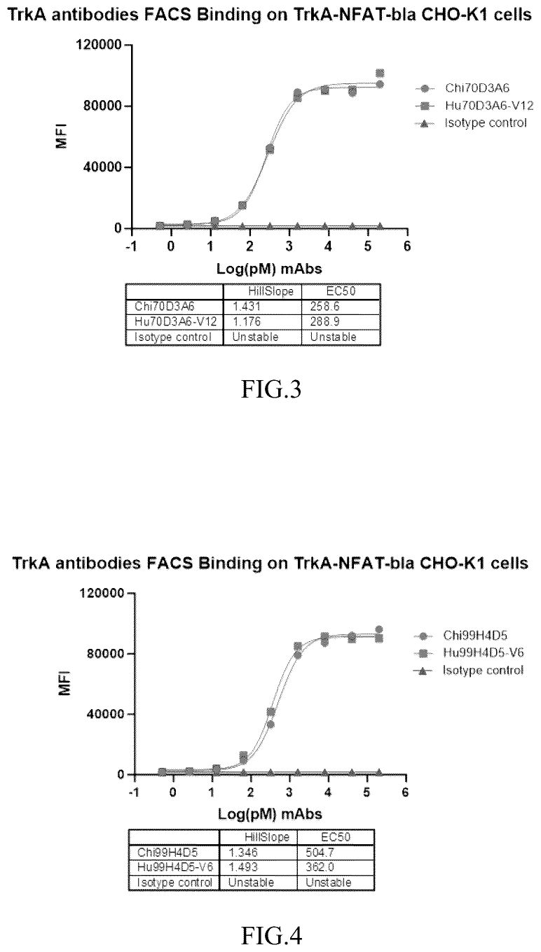

As shown in the FIG. 3, the EC50 value of Hu70D3A6-V12 in FACS binding analysis was about 288.9 pM, comparable to that of chi70D3A6 (the chimeric antibody). These results suggest that Hu70D3A6-V12 fully retained the binding activity of chi70D3A6.

As shown in the FIG. 4, the EC50 value of Hu99H4D5-V6 in FACS binding analysis was about 362.0 pM, comparable to that of chi99H4D5 (the chimeric antibody). These results suggest that Hu99H4D5-V6 fully retained the binding activity of chi99H4D5.

### Example 6 In Vitro Functional Analysis of the TrkA Antibodies

To evaluate the activities of the TrkA antibodies of the present disclosure, the activation of NGF-TrkA downstream signaling was assessed using CellSensor® TrkA-NFAT-bla CHO-K1 cells.

TrkA-activation dependent NFAT-promoter driven reporter assay following Invitrogen's CellSensor TrkA-NFAT-bla CHO-K1 cell-based assay protocol. Briefly, harvest cells and resuspend in Assay Medium to a density of 375, 000 cells/mL, then add Assay Medium or the cell suspension to each testing well. Incubate the plates in a 37 °C/5% CO₂ incubator for 16 hours. Add 8 μl of NGF or Diluted antibody samples to testing wells. Incubate the plate in a humidified 37 °C/5% CO₂ incubator for 5 hours. Prepare 6× LiveBLAzer™-FRET B/G (CCF4-AM) Substrate Mixture before use. Remove the assay plate from the incubator and allow the plate to equilibrate to room temperature. Add 8 μL of 6× Substrate Mixture to each well. Incubate at room temperature for 2 hours. At last, set the fluorescence plate reader to bottom-read mode, read the wells using a dual emission wavelength measurement protocol (measurement 1: 405 nm excitation & 460 nm emission; measurement 2: 405 nm excitation & 530 nm emission).

TrkA-activation dependent Akt phosphorylation by HTRF assay following Cisbio's pAKT(5473) assay manual. Cellsensor TrkA-NFAT-bla CHO-K1 cells (K1516, Life Technologies) were seeded in 96-well plates with 100 μl media on the day before the experiment. The cells were pre-incubated with 3-fold serial diluted antibody solution in a humidified 37 °C/5% CO₂ incubator for 30 minutes. Then cell supernatant was removed and 50 μl/well of 2×NGF was added. The plates were incubated in a 37 °C/5% CO₂ incubator for 30 minutes. Cell supernatant was removed carefully either by aspirating supernatant or by flicking the plate. 50 μl of supplemented lysis buffer (1×) was added immediately and incubated for 30 minutes at room temperature while shaking. After homogenization by pipetting up and down, 16 μl cell lysate was transferred from the 96-well plate to a 384-well plate. 16 μl of supplemented lysis buffer (1×) was dispensed to the Cell-free Control wells. 16 μl of homogenized cell lysate was dispensed to the Test Sample wells, the Unstimulated Control wells, and Stimulated Control wells were incubated for at least 4 hours at room temperature. The reader for Eu³⁺ Cryptate was set up and the fluorescence emission at two different wave-lengths (665 nm and 620 nm) was read on a compatible HTRF® reader. NGF or TrkA agonists dose response curve was analyzed with Four-Parameter Fit using GraphPad Prism software.

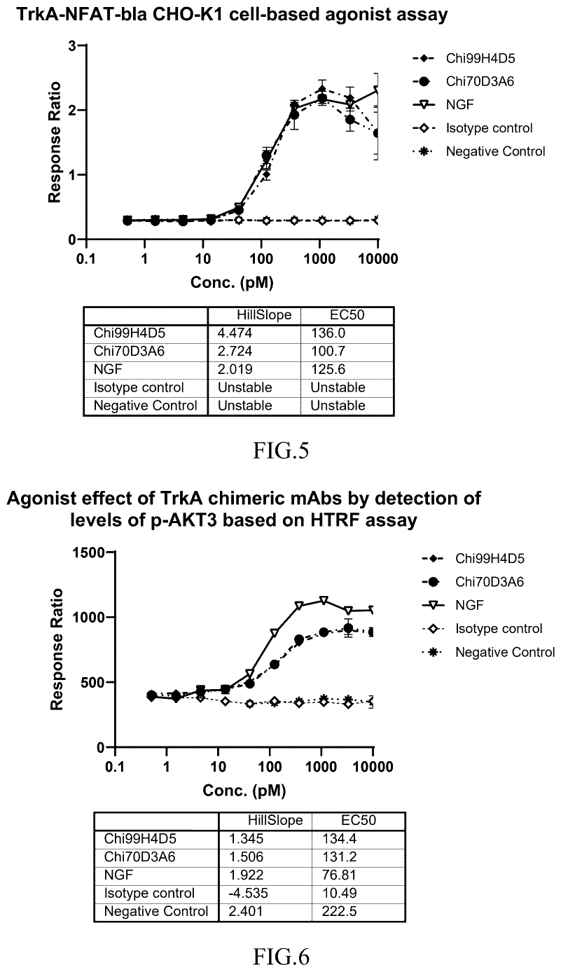

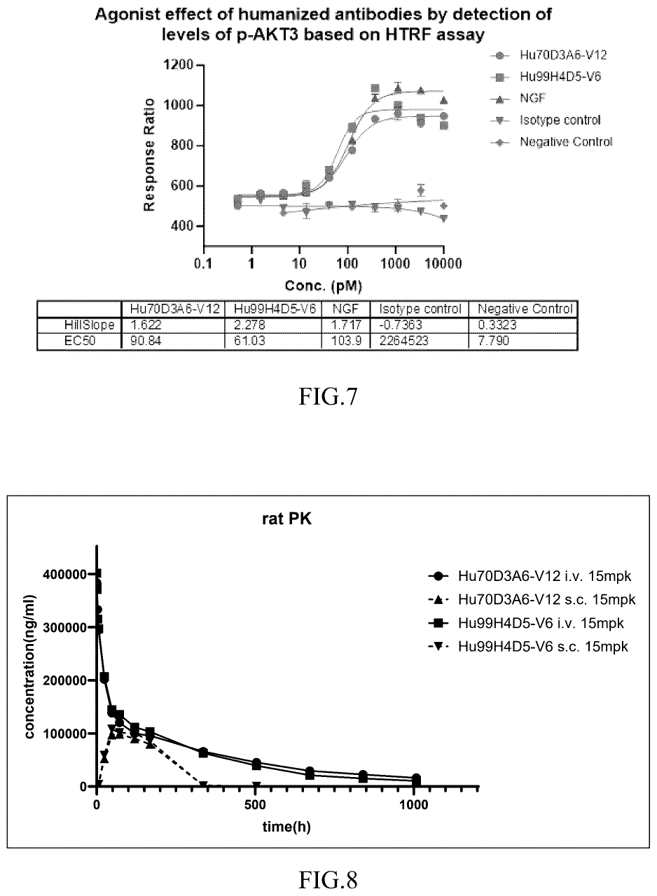

The results are summarized in FIGS. 5, 6, and 7. In the NFAT assay, the TrkA antibodies chi99H4D5 and chi70D3A6 activated the TrkA-PLC7 pathway with EC₅₀ values similar to those of NGF. In the HTRF assay, the EC₅₀ values of the four TrkA antibodies (chi99H4D5, chi70D3A6, Hu99H4D5-V6, and Hu70D3A6-V12) for activating the TrkA-AKT signaling pathway were also similar to those of NGF. These results suggest that the TrkA agonistic antibodies exhibit comparable potency to NGF in activating the TrkA signaling pathway.

### Example 7 the PK Study in Rat

As shown in FIG. 8 and Table 6, PK study were performed in male SD rat, n=3 in each timepoint in each group, the serum were collected and tested by ELISA method. The results demonstrated the longer half-life (T_{1/2, i.v.:} 189~335h) of TrkA agonistic antibody in rats.

TABLE 6
PK data in male SD rat

| | | Hu70D3A6- | Hu99H4D5- | Hu70D3A6- | Hu99H4D5- |
|---|---|---|---|---|---|
| | | V12 (iv | V6 (iv | V12 (sc | V6 (sc |
| PK Parameters | Unit | 15 mpk) | 15 mpk) | 15 mpk) | 15 mpk) |
| T_{1/2} | h | 335 | 189 | 198 | 70 |
| C₀ | μg/mL | 387 | 40 | | |
| Cₘₐₓ | μg/mL | | | 101 | 119 |
| AUC₀₋ₜ | h · μg/mL | 59877 | 56623 | 15713 | 16524 |
| AUC_{0-∞} | h · μg/mL | 67725 | 59511 | 35999 | 21235 |
| CL | mL/h/kg | 0.222 | 0.262 | | |
| Vss | mL/kg | 99.0 | 75.1 | | |
| MRT_{0-∞} | h | 447 | 305 | 337 | 156 |
| F (calculated by | % | | | 26 | 29 |
| AUC₀₋ₜ) | | | | | |

### Example 8 the NGF Blocking Assay

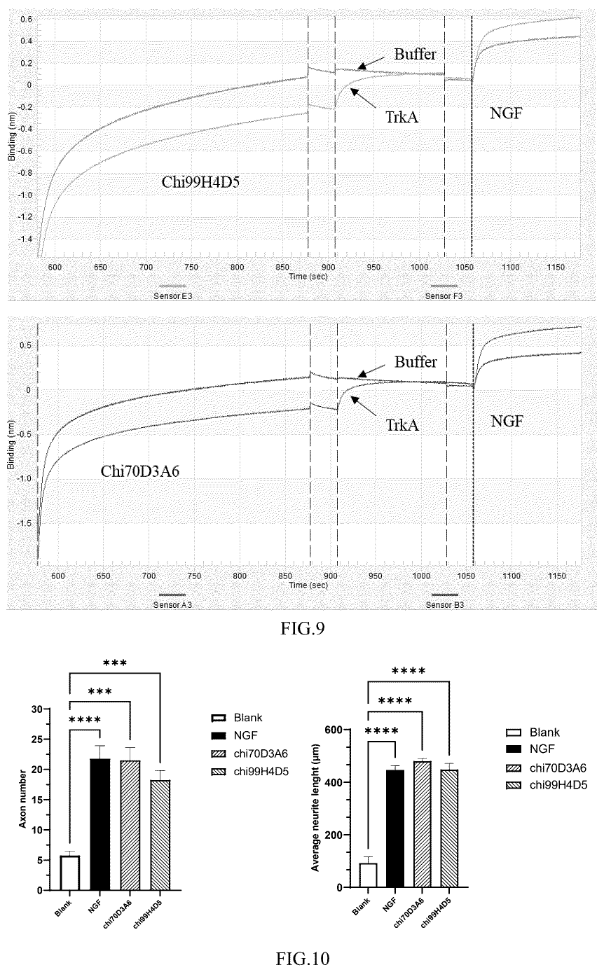

To determine whether TrkA antibodies can block the binding between TrkA and NGF. A bio-layer interferometry (BLI) method-based competition assay was performed on Octet Red384. Briefly, anti-human Fc(AHC) Biosensor (Fortebio) were put in PBS with 0.1% w/v bovine serum albumin and t0.05% Tween-20 (assay buffer) for a minimum of 10 min in the pre-wetting plate. The antibody at 100 nM in assay buffer was captured on the sensor to obtain a capture level of 0.5~1.5 nm. The loaded biosensors were then equilibrated in an assay buffer for a minimum of 15s followed by TrkA ECD binding for a minimum of 60s. After the second equilibration, NGF was loaded for a minimum of 60s. Antibodies Chi70D3A6 and Chi99H4D5 do not block the binding between TrkA and NGF (FIG. 9).

### Example 9 the Effects of the TrkA Antibody on DRG Neurons Survival and Axon Extension—the Neurotrophic Effect In Vitro

DRG dissociated primary neurons were prepared from humanized embryonic mice. DRG were collected, incubated for 5-8 mins at 37 °C with 0.05% Trypsin-EDTA, pipetted into single cells, and then plated onto a petri dish pretreated with poly-D-lysine and laminin, at a density of 5000 cells/well on a 24 well-plate with different DRG conditioned medium (NGF OnM; NGF 0.3 nM; Anti-TrkA antibodies 0.3 nM). The medium was half changed after 48 h, while 5-fluoro-2′-deoxyuridine at a final concentration of 5 μg/ml was added to inhibit the excessive proliferation of glial cells. After 72 h of DRG seeding, 10× and 20× brightfield photography were performed.

After 72 hours of DRG seeding, the intermediate visual field of each well of the experimental group was taken (20×), the axon path of DRG neurons was traced and the length was calculated by ImageJ, and the average length of each field of view was used for the above statistics. The graph was generated using GraphPad Software, and the data was reported as the mean±standard error of the mean (SEM). One-way ANOVA was used to analyze the data. *P<0.05, **P<0.01, ***P<0.001, ****P<0.001 compared with Blank.

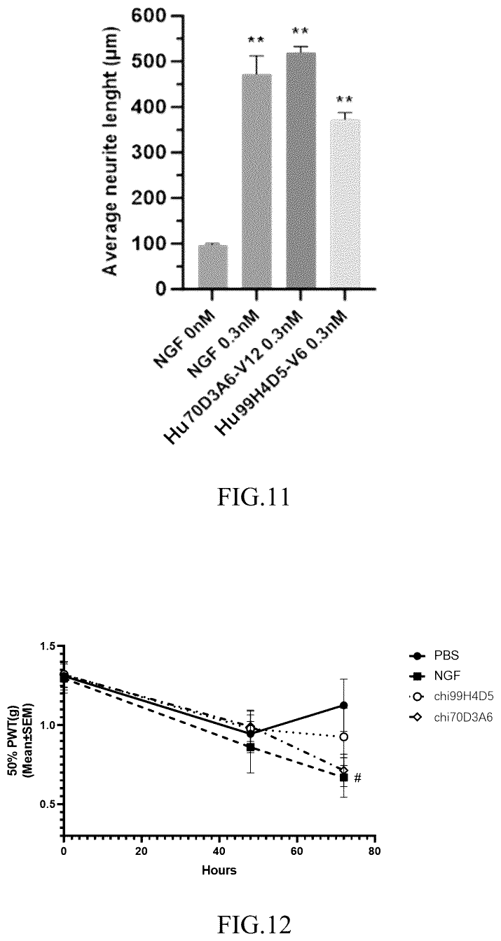

As shown in FIG. 10, the average axon length of 0.3 nM NGF group and 0.3 nM TrkA agonistic antibodies group exceeded 200 μm. As shown in FIG. 11, 0.3 nM NGF group and 0.3 nM TrkA agonistic antibodies group increased the the average neurite length significantly. These results indicate that Chi70D3A6 and Chi99H4D5, as well as Hu99H4D5-V6 and Hu70D3A6-Vl2 have the potential neurite outgrowth effect like NGF on survival and axon extension of the DRG neurons (all p<0.001 in axon number, all p<0.0001 in average neurite length).

### Example 10 the TrkA Agonistic Antibodies do not Elicit Mechanical Hyperalgesia In Vivo

NGF may increase sensitivity to noxious stimuli mediated by both TrkA and p75NTR. Such as the injection of exogenous NGF produces mechanical and thermal hypersensitivity changes in rodents and humans.

To understand whether the TrkA agonist antibody can induce nociceptor sensitization, the behavioral response of adult mice to mechanical stimuli after a single intraplantar injection of either NGF or TrkA agonist antibodies was measured.

Mechanical withdrawal thresholds were assessed by applying calibrated von Frey monofilaments (0.02e1 g, North Coast Medical Inc.) to the plantar surface of the hind paw. 50% PWT was determined according to the “up-down” method.

Before the study, mice were acclimated to the laboratory environment for 60 minutes daily over three days. Baseline measurements were then taken. Mice with 50% paw withdrawal threshold (PWT) greater than 1 g were randomly divided into groups (n=7-8). Each group received an intraplantar injection of either PBS, NGF, or TrkA agonistic antibodies at a dose of 6 μM in 25 μL per mouse. The 50% PWT was measured at 48 and 72 hours post-injection.

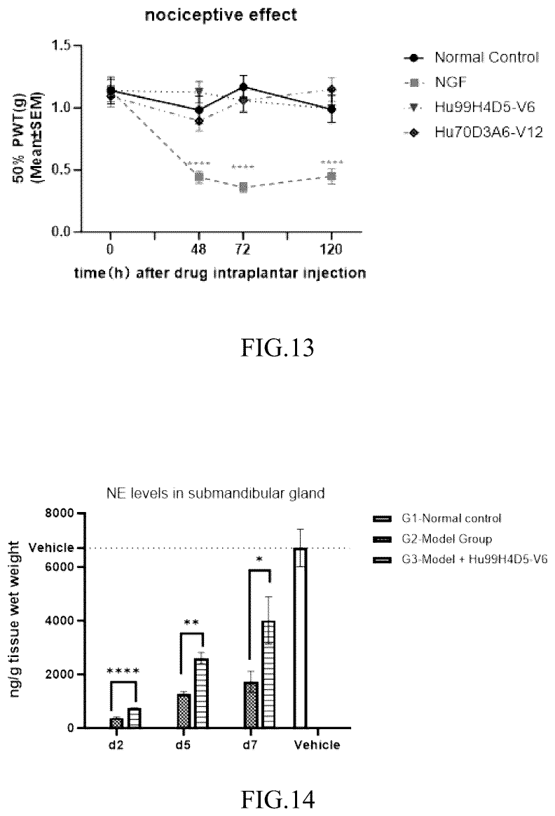

As shown in FIG. 12 and FIG. 13, NGF injection significantly induced mechanical allodynia. The 50% PWT in the NGF group decreased significantly at 48 hours and at 72 hours compared to baseline, with mechanical hypersensitivity lasting over 120 hours. This reduction was significant compared to the PBS group (p<0.05). In contrast, TrkA agonist antibodies induced less or no mechanical allodynia compared to NGF. In conclusion, unlike NGF, TrkA agonist antibodies do not trigger significant injection site pain or hyperalgesia.

### Example 11 the Neurotrophic Effect In Vivo

Activation of NGF-TrkA signaling resulted in a strong stimulating effect on the regeneration of fully developed adrenergic neurons in the peripheral tissues after axotomy. The 6-OHDA sympathectomy model is utilized to assess the efficacy of compounds in promoting the repair of sympathetic nerves. It has been reported that the NGF/TrkA pathway generates neurotrophic effects in this model. To assess the in vivo neurotrophic effects of the TrkA agonist antibody in this model, a subcutaneous injection of the TrkA agonist (30 mg/kg) was administered 2 days prior to inducing the 6-OHDA lesion (intraperitor injection with 60 mg/kg). The neurotrophic effect of the TrkA agonist antibody was further evaluated at 2, 5, and 7 days post-6-OHDA injection, using endogenous NE levels in the submandibular gland as a readout.

As illustrated in FIG. 14, the endogenous NE levels in tissues experienced a notable depletion, dropping to approximately 5% two days after 6-OHDA administration compared to age- and sex-matched normal untreated mice (Vehicle). Over subsequent days, there was a progressive improvement, with levels reaching about 19% after 5 days and approximately 25% after 7 days. When comparing to the model group at various time points, administration of the TrkA agonist yielded a significant increase in endogenous NE levels (p<0.05). The present findings demonstrate that the TrkA agonist antibody effectively protects peripheral noradrenergic nerves in the 6-OHDA lesion model.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. An isolated antibody or an antigen-binding fragment thereof, which is capable of specifically binding to TrkA, said antibody or the antigen-binding fragment thereof has one or more properties selected from the group consisting of:
1) said antibody or the antigen-binding fragment thereof is a TrkA agonistic antibody or the antigen-binding fragment thereof,
2) being capable of binding with TrkA receptor and selectively activating the downstream signal pathway and avoiding p75 binding and its mediated biological effect;
3) being capable of promoting neuron growth and recovery in vitro and in vivo;
4) being capable of improving drug-like properties over NGF with longer half-life and/or less injection frequency;
5) not substantially blocking the binding between TrkA and NGF;
6) not substantially competing with NGF for binding to TrkA;
7) being capable of treating disease or disorder with less pain or irritation compared with NGF.

2-3. (canceled)

4. The antibody or the antigen-binding fragment of claim 1, which is capable of competing with a reference antibody for binding to said TrkA, wherein said reference antibody comprises LCDR1-3 and HCDR1-3, said LCDR1 comprises the amino acid sequence as set forth in SEQ ID NO: 11, said LCDR2 comprises the amino acid sequence as set forth in SEQ ID NO: 12 (RT), said LCDR3 comprises the amino acid sequence as set forth in SEQ ID NO: 47, said HCDR1 comprises the amino acid sequence as set forth in SEQ ID NO: 43, said HCDR2 comprises the amino acid sequence as set forth in SEQ ID NO: 44, and said HCDR3 comprises the amino acid sequence as set forth in SEQ ID NO: 45.

5. The antibody or the antigen-binding fragment of claim 1, which comprises at least one of HCDR1-3 of a heavy chain variable region (VH), and said VH comprises an amino acid sequence as set forth in SEQ ID NO: 54.

6-11. (canceled)

12. The antibody or the antigen-binding fragment of claim 1, which comprises HCDR1, HCDR2 and HCDR3, said HCDR1 comprises an amino acid sequence as set forth in SEQ ID NO: 43, said HCDR2 comprises an amino acid sequence as set forth in SEQ ID NO: 44, and said HCDR3 comprises an amino acid sequence as set forth in SEQ ID NO: 45.

13. The antibody or the antigen-binding fragment of claim 1, which comprises a heavy chain variable region (VH), and said heavy chain variable region comprises an amino acid sequence as set forth in SEQ ID NO: 54.

14. The antibody or the antigen-binding fragment of claim 1, which comprises a heavy chain variable region (VH), and said heavy chain variable region comprises an amino acid sequence as set forth in any one of SEQ ID NO: 7, SEQ ID NO: 8, SEQ ID NO: 9, SEQ ID NO: 10, SEQ ID NO: 17, SEQ ID NO: 18, SEQ ID NO: 50 and SEQ ID NO: 52.

15-20. (canceled)

21. The antibody or the antigen-binding fragment of claim 1, which comprises LCDR1, LCDR2 and LCDR3, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 47.

22. The antibody or the antigen-binding fragment of claim 1, which comprises a light chain variable region (VL), and said light chain variable region comprises an amino acid sequence as set forth in SEQ ID NO: 55.

23. The antibody or the antigen-binding fragment of claim 1, which comprises a light chain variable region, and said light chain variable region comprises an amino acid sequence as set forth in any one of SEQ ID NO: 14, SEQ ID NO: 20, SEQ ID NO: 51 and SEQ ID NO: 53.

24-25. (canceled)

26. The antibody or the antigen-binding fragment of claim 1, comprising HCDR1-3 and LCDR1-3, wherein the HCDR1-3 and LCDR1-3 comprises the amino acid sequences selected from the group consisting of:
1) said HCDR1 comprises an amino acid sequence as set forth in SEQ ID NO: 1, said HCDR2 comprises an amino acid sequence as set forth in SEQ ID NO: 2, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 13;
2) said HCDR1 comprises an amino acid sequence as set forth in SEQ ID NO: 1, said HCDR2 comprises an amino acid sequence as set forth in SEQ ID NO: 3, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 13;
3) said HCDR1 comprises an amino acid sequence as set forth in SEQ ID NO: 1, said HCDR2 comprises an amino acid sequence as set forth in SEQ ID NO: 4, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 13;
4) said HCDR1 comprises an amino acid sequence as set forth in SEQ ID NO: 1, said HCDR2 comprises an amino acid sequence as set forth in SEQ ID NO: 5, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 13;
5) said HCDR1 comprises an amino acid sequence as set forth in SEQ ID NO: 15, said HCDR2 comprises an amino acid sequence as set forth in SEQ ID NO: 3, said HCDR3 comprises an amino acid sequence as set forth in SEQ ID NO: 16, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 19;
6) said HCDR1 comprises an amino acid sequence as set forth in SEQ ID NO: 15, said HCDR2 comprises an amino acid sequence as set forth in SEQ ID NO: 5, said HCDR3 comprises an amino acid sequence as set forth in SEQ ID NO: 16, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 19; or
7) said HCDR1 comprises an amino acid sequence as set forth in SEQ ID NO: 15, said HCDR2 comprises an amino acid sequence as set forth in SEQ ID NO: 5, said HCDR3 comprises an amino acid sequence as set forth in SEQ ID NO: 6, said LCDR1 comprises an amino acid sequence as set forth in SEQ ID NO: 11, said LCDR2 comprises an amino acid sequence as set forth in SEQ ID NO: 12 (RT), and said LCDR3 comprises an amino acid sequence as set forth in SEQ ID NO: 19.

27. The antibody or the antigen-binding fragment of claim 1, comprising VH and VL, said VH and VL comprises amino acid sequences selected from the group consisting of:
1) said VH comprises an amino acid sequence as set forth in SEQ ID NO: 7, said VL comprises an amino acid sequence as set forth in SEQ ID NO: 14;
2) said VH comprises an amino acid sequence as set forth in SEQ ID NO: 8, said VL comprises an amino acid sequence as set forth in SEQ ID NO: 14;
3) said VH comprises an amino acid sequence as set forth in SEQ ID NO: 9, said VL comprises an amino acid sequence as set forth in SEQ ID NO: 14;
4) said VH comprises an amino acid sequence as set forth in SEQ ID NO: 10, said VL comprises an amino acid sequence as set forth in SEQ ID NO: 14;
5) said VH comprises an amino acid sequence as set forth in SEQ ID NO: 17, said VL comprises an amino acid sequence as set forth in SEQ ID NO: 20;
6) said VH comprises an amino acid sequence as set forth in SEQ ID NO: 18, said VL comprises an amino acid sequence as set forth in SEQ ID NO: 20;
7) said VH comprises an amino acid sequence as set forth in SEQ ID NO: 50, said VL comprises an amino acid sequence as set forth in SEQ ID NO: 51; or
8) said VH comprises an amino acid sequence as set forth in SEQ ID NO: 52, said VL comprises an amino acid sequence as set forth in SEQ ID NO: 53.

28. A fusion protein, comprising the antibody or the antigen-binding fragment of claim 1.

29. A protein conjugate, comprising the antibody or the antigen-binding fragment of claim 1.

30. Isolated nucleic acid molecule or molecules, encoding for the antibody or the antigen-binding fragment of claim 1.

31. Vector or vectors, comprising the isolated nucleic acid molecule or molecules of claim 30.

32. A cell, comprising the isolated nucleic acid molecule or molecules of claim 30.

33. A composition, comprising the antibody or the antigen-binding fragment of claim 1, and optionally a pharmaceutically acceptable excipient.

34-40. (canceled)

41. A method for preventing and/or treating a disorder or disease in a subject in need thereof, comprising administering to said subject an effective amount of the antibody or the antigen-binding fragment of claim 1.

42. The method of claim 41, wherein said disease or disorder comprise tumors, peripheral neuropathies, ophthalmic diseases, diabetes and related complications, cardiovascular disease and/or Alzheimer's disease.

43-52. (canceled)

53. A method for producing the antibody or the antigen-binding fragment of claim 1, comprising culturing the cell of claim 32 under conditions enabling expression of the antibody or the antigen-binding fragment of claim 1.
